(19) United States
(12) Patent Application Publication
(10) Pub. No.: US 2016/0249542 A1
(43) Pub. Date: Sep. 1, 2016

(54) SELF-REPRODUCING HYBRID PLANTS

(71) Applicant: PIONEER HI-BRED INTERNATIONAL INC., Johnston, IA (US)

(72) Inventors: SHAI JOSHUA LAWIT, URBANDALE, IA (US); MARTA CIFUENTES OCHOA, WEST MOINES, IA (US); MARISSA KATHARINE SIMON, GRIMES, IA (US)

(21) Appl. No.: 15/030,471

(22) PCT Filed: Oct. 28, 2014
(86) PCT No.: PCT/US2014/062633

§ 371 Date: Apr. 19, 2016

Related U.S. Application Data
(60) Provisional application No. 61/897,058, filed on Oct. 29, 2013.

(51) Int. Cl. A01H 1/02

(52) U.S. Cl. CPC A01H 1/02

(57) ABSTRACT

Compositions and methods for the production of self-reproducing hybrid plants are provided. Compositions include suppression cassettes encoding polynucleotides and promoters that result in the MiMe clonal diploid gamete phenotype compositions and suppression cassettes and expression cassettes useful for genome elimination of a parental diploid gamete in a fertilized zygote. The methods involve crossing a first plant comprising a first suppression cassette responsible for producing the MiMe clonal diploid gamete phenotype and a first expression cassette expressing an active CENH3 mutant with a second plant comprising a second suppression cassette that reduces the level of wild-type CENH3 and a second expression cassette comprising a polynucleotide expressing CENH3 specifically in the ovule. Self fertilization of the resultant progeny plant results in the elimination of the male diploid genome in the zygote and normal development of the endosperm. Additionally provided are plants and seeds produced by the methods of the invention.

### FIELD OF THE INVENTION

The invention relates to the field of genetic manipulation of plants, particularly the production of self-reproducing hybrid plants.

### BACKGROUND OF THE INVENTION

Although plant breeding programs worldwide have made considerable progress developing new cultivars with improved disease resistances, yields and other useful traits, breeding as a whole relies on screening numerous plants to identify novel, desirable characteristics. Very large numbers of progeny from crosses often must be grown and evaluated over several years in order to select one or a few plants with a desired combination of traits.

A continuing goal of plant breeders is to develop stable, high-yielding varieties that are agronomically sound. Standard breeding of diploid plants often requires screening and back-crossing of a large number of plants to achieve the desired genotype. One solution to the problem of screening large numbers of progeny has been to generate doubled haploid plants that eliminate genomic heterogeneity and, thus, any segregation of traits. When economically and biologically feasible, additional gains are often made through employing heterosis with hybrids of two inbred parents.

Heterosis studies in soybean estimate that there is approximately a 10% yield improvement potential with hybrids. However, hybrid soybeans have never been developed because pollen flow from male to female inbreds is very poor. Pollen vectoring is a problem that has few, if any, solutions available for high volume hybrid production in soybean. However, hand crosses could produce limited hybrid numbers and volume production of hybrid soybean could commence with the aid of self-reproduction.

Furthermore, current transgene introgression requires the maintenance of transgene homozygosity in inbred lines and varieties, which greatly limits the potential for native and transgene trait stacking. However, by using hybrid plants, transgenes could be stacked much more easily by providing a single copy from each parent. Availability of a system to generate self-reproducing hybrids would find value in both plant breeding and development.

Thus, marked improvements in the economics of breeding can be achieved via self-reproducing hybrid production, since selection and other procedural efficiencies can be substantially improved. Current methods for parent-specific genome elimination result in plants with near total male sterility and very low rates of female fecundity, making propagation of the hybrid plant difficult.

### BRIEF SUMMARY OF THE INVENTION

Compositions and methods for the production of self-reproducing hybrid plants are provided. Compositions include suppression cassettes encoding polynucleotides and promoters that result in the MiMe diploid clonal gamete phenotype. Further provided are methods and compositions comprising suppression cassettes and expression cassettes resulting in genome elimination of a parental diploid gamete in the fertilized zygote, producing a self-reproducing hybrid plant.

Methods for producing a self-reproducing hybrid plant include crossing a first plant comprising a first suppression cassette responsible for producing the MiMe diploid clonal gamete phenotype and a first expression cassette expressing an active CENH3 mutant with a second plant comprising a second suppression cassette that reduces the level of wild-type CENH3 and a second expression cassette comprising a polynucleotide expressing CENH3 specifically in the ovule. Self fertilization of the resultant progeny plant results in the elimination of the male diploid genome in the zygote and normal development of the endosperm. Additionally provided are plants and seeds, particularly hybrid plants and hybrid seeds, produced by the methods of the invention.

### BRIEF DESCRIPTION OF THE DRAWINGS

FIG. 1 shows a transgene system designed to activate clonal reproduction in hybrids using female genome elimination in the zygote, but maintain normal sexual reproduction in the parental inbred varieties. Upon hybrid creation through crossing of the two parent lines, Transactivator B (for example) drives constitutive suppression of Meiosis genes leading to unreduced clonal gametes. Transactivator A (for example) drives suppression of CENH3 in the ovule, setting the stage for the CENH3 GFP-tailswap expression. An ovule promoter drives expression of the CENH3 GFP-tailswap in the ovule leading to female genome elimination in the first zygotic mitosis. A central cell promoter drives the WT CENH3 in the central cell allowing normal mitosis in the endosperm, and preventing female genome elimination in the endosperm.

FIG. 2 shows an example of the transgene system designed to activate clonal reproduction in hybrids using female genome elimination in the embryo, but maintain normal sexual reproduction in the parental inbred varieties. T7 polymerase and GaI4DBD-VP16 (or LexA-CBF) two component activation systems are shown as examples of possible transactivators that would activate the self reproduction system only once brought together in a hybrid cross containing the two transgene cassettes where the amiRNA silencing elements would be activated. Specifically, T7 polymerase (for example) drives constitutive suppression of Meiosis (MiMe) genes leading to unreduced clonal gametes. GaI4DBD-VP16 (for example) drives suppression of CENH3 in the ovule, setting the stage for the CENH3 GFP-tailswap expression. An ovule promoter drives expression of the CENH3 GFP-tailswap leading to female genome elimination in the first zygotic mitosis.AT-DD65 PRO drives the WT CENH3 in the central cell allowing normal mitosis in the endosperm and preventing female genome elimination in the endosperm.

FIG. 3 shows the mechanisms utilized to result in self-reproducing hybrid plants using female genome elimination. For example, an apomeiosis system (e.g. MiMe) produces unreduced gametes. Expression of genome elimination technology occurs in the egg cell. Fertilization leads to a 4n zygote and 6n endosperm (4m:2p). Genome elimination of the egg cell genome in the zygote leads to a 2n (paternal genome) zygote/embryo. Normal endosperm develops from a 4m:2p genome which has the proper 2m:1p genome ratio.

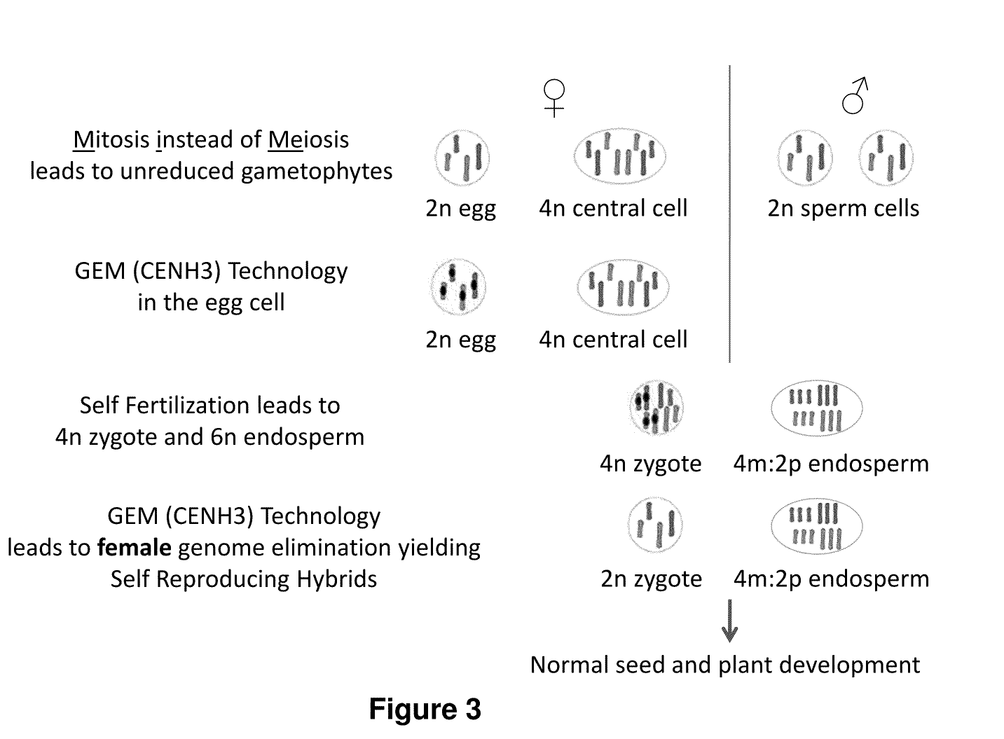

FIG. 4 shows (Left) quadruply labeled embryo sac in an ovule from Arabidopsis transgenic PHP47078 at the egg cell stage of development. These labeled embryo sac cells allow cell development and viability to be monitored. (Right) Triply labeled embryo sac in an ovule from Arabidopsis transgenic PHP42551. This embryo sac is at the early embryo stage of development prior to the globular stage. Numerous endosperm nuclei are visible in cyan demonstrating the ability to follow early endosperm development.

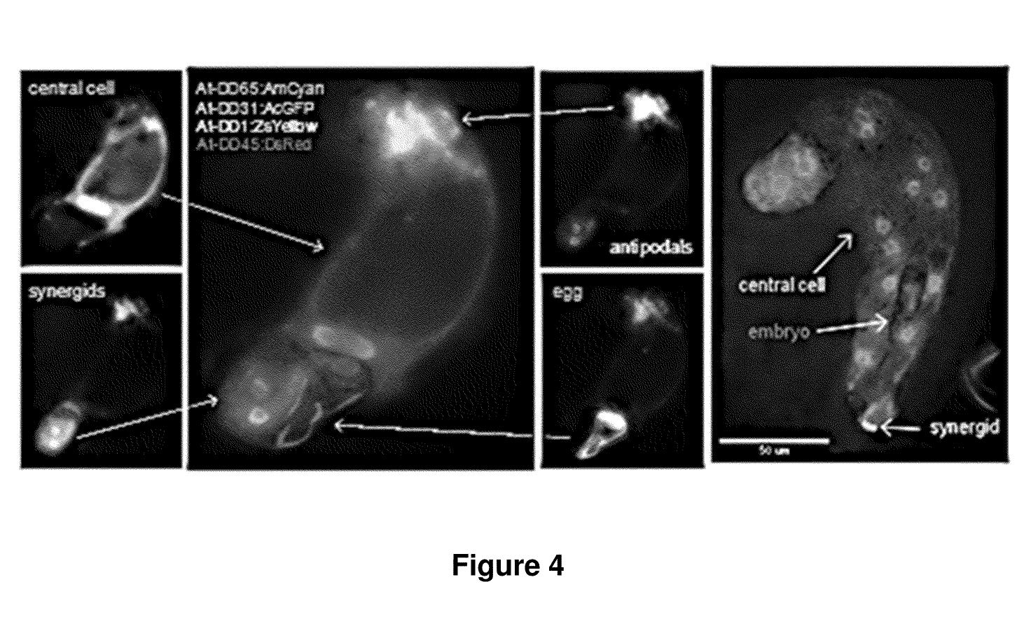

FIG. 5 shows an example of the transgene system designed to activate clonal reproduction in hybrids using male genome elimination in the embryo, but maintain normal sexual reproduction in the parental inbred varieties. Two component activation (transactivator) systems are shown as examples of possible transactivators that would activate the self reproduction system only once brought together in a hybrid cross containing the two transgene cassettes where the amiRNA silencing elements would be activated. Upon hybrid creation through crossing of the two parent lines, Transactivator B drives constitutive suppression of Meiosis (MiMe) genes leading to unreduced clonal gametes. Transactivator A drives suppression of CENH3 in the cells undergoing meiosis and through a few subsequent mitotic divisions, setting the stage for the CENH3 GFP-tailswap expression. An egg cell promoter drives the WT CENH3 in the egg cell enabling male genome elimination in the first zygotic mitosis. A pollen or sperm cell promoter drives expression of the CENH3 GFP-tailswap in the sperm cell leading to male genome elimination in the first zygotic mitosis. A central cell promoter drives the CENH3 GFP-tailswap in the central cell allowing normal mitosis in the endosperm, and preventing female genome elimination in the endosperm (no CENH3 parental conflict).

FIG. 6 shows an example of the transgene system designed to activate clonal reproduction in hybrids using female genome elimination in the embryo, but maintain normal sexual reproduction in the parental inbred varieties. Transactivator A drives constitutive suppression of Meiosis (MiMe) genes leading to unreduced clonal gametes. tetR(for example) represses the native CENH3 in the female germline, setting the stage for the CENH3 GFP-tailswap expression. For this to occur the CENH3 native promoter must be modified through homologous recombination or another targeted gene replacement technology. Alternatively, the native CENH3 may be knocked-out or silenced, and a transgenic copy of the CENH3 is controlled by a controllable repressor. An egg cell promoter drives expression of the CENH3 GFP-tailswap in the egg cell leading to female genome elimination in the first zygotic mitosis. A central cell promoter drives the WT CENH3 in the central cell allowing normal mitosis in the endosperm, and preventing female genome elimination in the endosperm. Following the F1 production from the two parent lines, the two-component transcriptional activator and repressor system are brought into a common hybrid genome and activate the silencing elements and/or repress the genes required for MiMe and genome elimination.

FIG. 7 shows an example of the transgene system designed to activate clonal reproduction in hybrids using male genome elimination in the embryo, but maintain normal sexual reproduction in the parental inbred varieties. Transactivator A drives constitutive suppression of Meiosis (MiMe) genes leading to unreduced clonal gametes. tetR (for example) represses expression of the native CENH3 in the cells undergoing meiosis and through a few subsequent mitotic divisions, setting the stage for the CENH3 GFP-tailswap expression. For this to occur, the CENH3 native promoter must be modified through homologous recombination or another targeted gene replacement technology. Alternatively, the native CENH3 may be knocked-out or silenced, and a transgenic copy of the CENH3 is controlled by a controllable repressor. An egg cell promoter drives the WT CENH3 in the egg cell enabling male genome elimination in the first zygotic mitosis. A pollen or sperm cell promoter drives expression of the CENH3 GFP-tailswap in the sperm cell leading to male genome elimination in the first zygotic mitosis. A central cell promoter drives the CENH3 GFP-tailswap in the central cell allowing normal mitosis in the endosperm, and preventing female genome elimination in the endosperm (no CENH3 parental conflict). Following the F1 production from the two parent lines, the two-component transcriptional activators are brought into a common hybrid genome and activate the silencing elements required for MiMe and genome elimination.

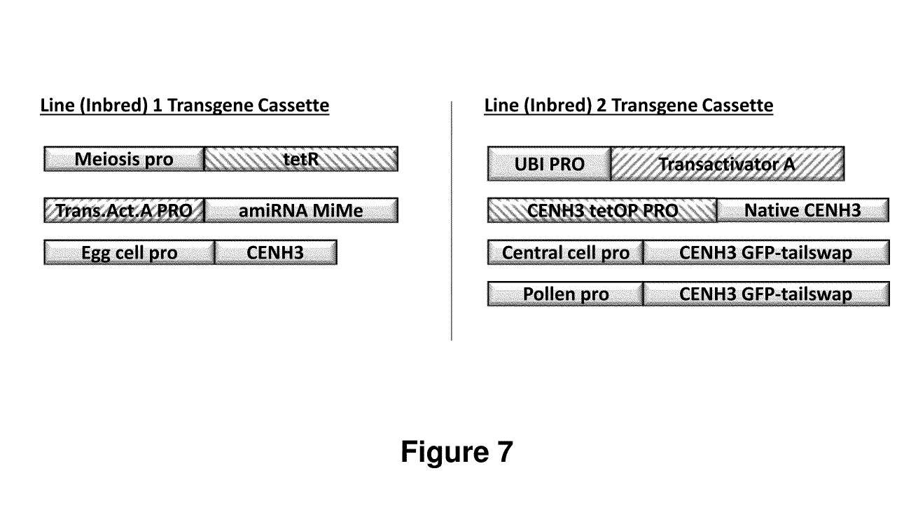

FIG. 8 shows the mechanisms utilized to result in self-reproducing hybrid plants using male genome elimination. For example, an apomeiosis system (e.g. MiMe) produces unreduced clonal gametes. Expression of genome elimination technology occurs in the central cell and sperm cells. Fertilization leads to a 4n zygote and 6n endosperm (4m:2p). Genome elimination of the sperm cell genome in the zygote leads to a 2n (maternal genome) zygote/embryo. Normal endosperm develops from a 4m:2p genome which has the proper 2m:1p genome ratio

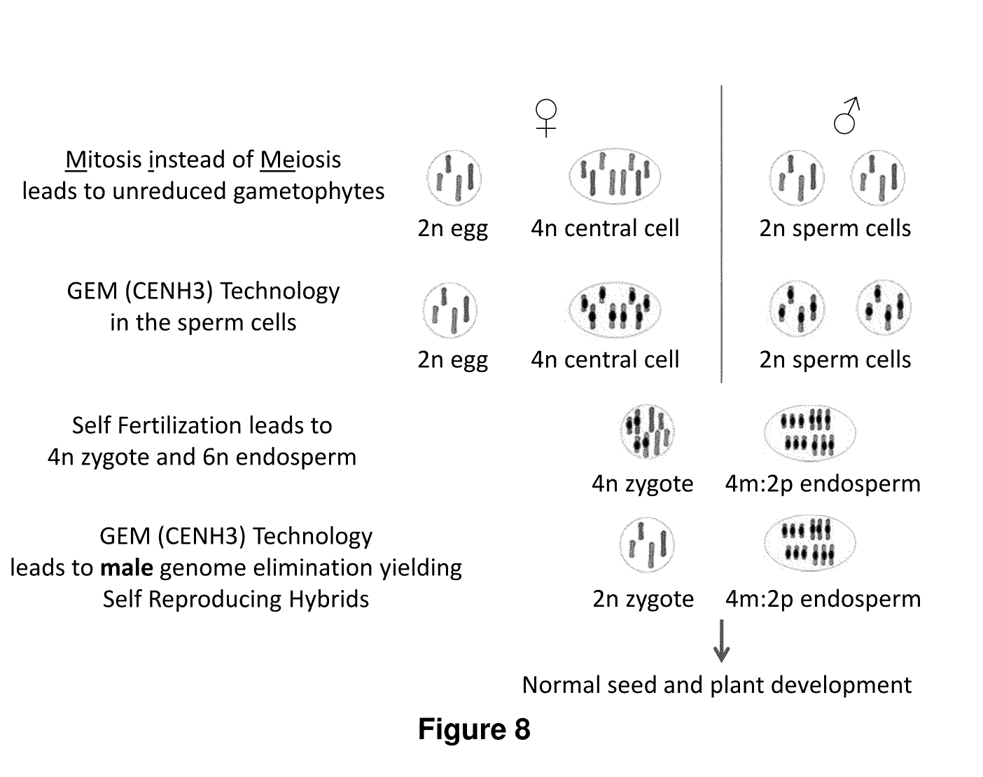

FIG. 9 shows a DAPI stained chromosome spread of a first (A) and second meiotic division in male meiocytes from Arabidopsis amiRNA construct targeting PRD3 (PHP73406). Univalents segregate randomly due to the lack of double strand breaks.

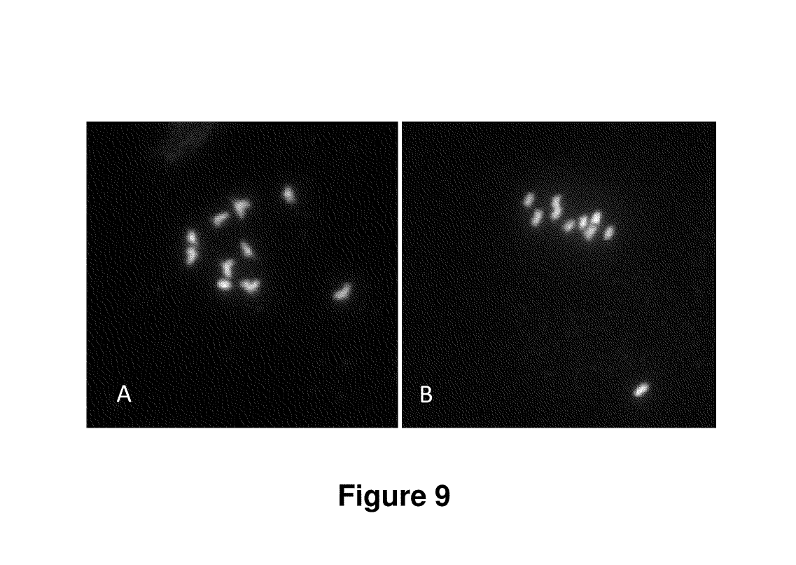

FIG. 10 shows a DAPI stained chromosome spread of a first (A) and second (B) meiotic division in male meiocytes from Arabidopsis amiRNA construct targeting REC8 (PHP72993). Fragmentation of chromosomes occurs during meiosis leading to unviable gametes.

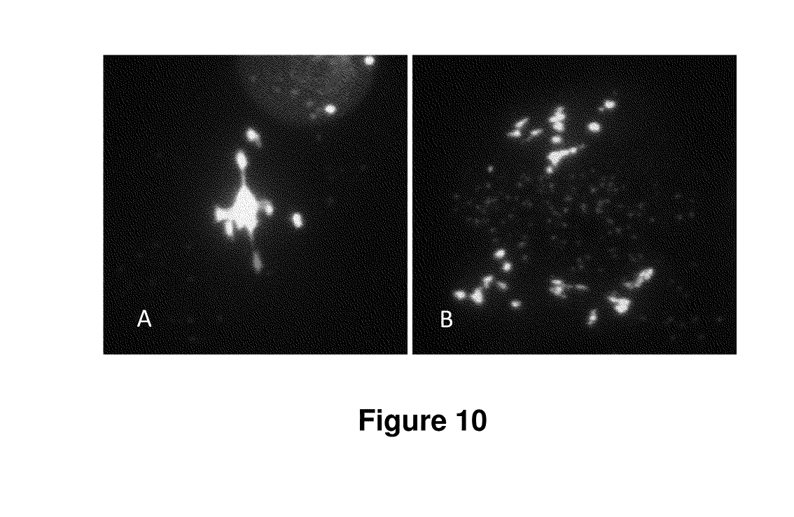

FIG. 11 shows the ploidy content of a wild type (diploid) soy plant and the tetraploid offspring of an amiRNA construct targeting OSD1 which produced diploid gametes in both male and female organs in soy.

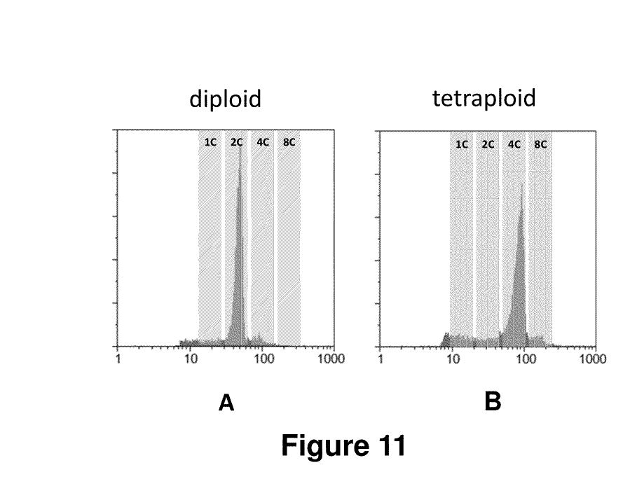

FIG. 12 (left) shows the DNA content (ploidy) of nuclei from a haploid Arabidopsis plant generated from the cross of pollen from a plant expressing suppression sequences no. 279 and 280 (a CENH3 amiRNA) in combination with expression of an active CENH3 tailswap polypeptide onto a WT female plant. (Center) Shows the DNA content (ploidy) of nuclei a WT diploid Arabidopsis plant. (Right) Shows the DNA content (ploidy) of nuclei from a tetraploid T2 plant expressing an Osd1 amiRNA.

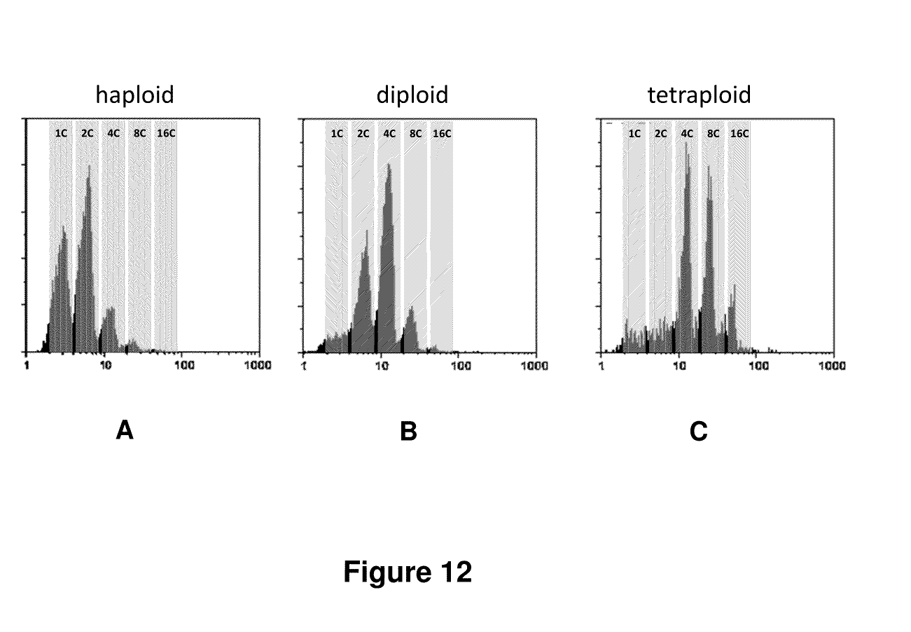

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

TABLE 1

| | | | POLYNUCLEOTIDE/ |
|---|---|---|---|
| SEQ ID. | ORGANISM NAME | DESCRIPTION | POLYPEPTIDE (PN/PP) |
| SEQ ID NO: 1 | ARTIFICIAL SEQUENCE | CONSERVED | PP |
| | | DOMAIN | |
| SEQ ID NO: 2 | ARABIDOPSIS THALIANA | SPO11-1 | PN |
| SEQ ID NO: 3 | ARABIDOPSIS THALIANA | 3ISD | PN |
| SEQ ID NO: 4 | ARABIDOPSIS THALIANA | REC8 | PN |
| SEQ ID NO: 5 | ARABIDOPSIS THALIANA | CENH3 | PN |
| SEQ ID NO: 6 | ARTIFICIAL SEQUENCE | PRIMER | PN |
| SEQ ID NO: 7 | ARTIFICIAL SEQUENCE | PRIMER | PN |
| SEQ ID NO: 8 | ARTIFICIAL SEQUENCE | PRIMER | PN |
| SEQ ID NO: 9 | ARTIFICIAL SEQUENCE | PRIMER | PN |
| SEQ ID NO: 10 | ARTIFICIAL SEQUENCE | PRIMER | PN |
| SEQ ID NO: 11 | ARTIFICIAL SEQUENCE | PRIMER | PN |
| SEQ ID NO: 12 | ARTIFICIAL SEQUENCE | PRIMER | PN |
| SEQ ID NO: 13 | BRASSICA NAPUS | CENH3 | PN |
| SEQ ID NO: 14 | BRASSICA RAPA | CENH3 | PN |
| SEQ ID NO: 15 | BRASSICA RAPA | CENH3 | PP |
| SEQ ID NO: 16 | GLYCINE MAX | CENH3 | PN |
| SEQ ID NO: 17 | GLYCINE MAX | CENH3 | PN |
| SEQ ID NO: 18 | MEDICAGO TRUNCATULA | CENH3 | PN |
| SEQ ID NO: 19 | MEDICAGO TRUNCATULA | CENH3 | PP |
| SEQ ID NO: 20 | ORYZA SATIVA | CENH3 | PN |
| SEQ ID NO: 21 | ORYZA SATIVA | CENH3 | PP |
| SEQ ID NO: 22 | ORYZA SATIVA | CENH3 | PN |
| SEQ ID NO: 23 | ORYZA SATIVA | CENH3 | PP |
| SEQ ID NO: 24 | SETARIA ITALICA | CENH3 | PN |
| SEQ ID NO: 25 | SETARIA ITALICA | CENH3 | PP |
| SEQ ID NO: 26 | SORGHUM BICOLOR | CENH3 | PN |
| SEQ ID NO: 27 | SORGHUM BICOLOR | CENH3 | PP |
| SEQ ID NO: 28 | VITIS VINIFERA | CENH3 | PN |
| SEQ ID NO: 29 | VITIS VINIFERA | CENH3 | PP |
| SEQ ID NO: 30 | ZEA MAYS | CENH3 | PN |
| SEQ ID NO: 31 | ZEA MAYS | CENH3 | PP |
| SEQ ID NO: 32 | BRASSICA NAPUS | OSD1 | PN |
| SEQ ID NO: 33 | BRASSICA RAPA | OSD1 | PN |
| SEQ ID NO: 34 | BRASSICA RAPA | OSD1 | PP |
| SEQ ID NO: 35 | BRASSICA RAPA | OSD1 | PN |
| SEQ ID NO: 36 | BRASSICA RAPA | OSD1 | PP |
| SEQ ID NO: 37 | GLYCINE MAX | OSD1 | PN |
| SEQ ID NO: 38 | GLYCINE MAX | OSD1 | PP |
| SEQ ID NO: 39 | GLYCINE MAX | OSD1 | PN |
| SEQ ID NO: 40 | GLYCINE MAX | OSD1 | PP |
| SEQ ID NO: 41 | MEDICAGO TRUNCATULA | OSD1 | PN |
| SEQ ID NO: 42 | MEDICAGO TRUNCATULA | OSD1 | PP |
| SEQ ID NO: 43 | ORYZA SATIVA | OSD1 | PN |
| SEQ ID NO: 44 | ORYZA SATIVA | OSD1 | PP |
| SEQ ID NO: 45 | ORYZA SATIVA | OSD1 | PN |
| SEQ ID NO: 46 | ORYZA SATIVA | OSD1 | PP |
| SEQ ID NO: 47 | SORGHUM BICOLOR | OSD1 | PN |
| SEQ ID NO: 48 | SORGHUM BICOLOR | OSD1 | PP |
| SEQ ID NO: 49 | VITIS VINIFERA | OSD1 | PN |
| SEQ ID NO: 50 | VITIS VINIFERA | OSD1 | PP |
| SEQ ID NO: 51 | ZEA MAYS | OSD1 | PN |
| SEQ ID NO: 52 | ZEA MAYS | OSD1 | PP |
| SEQ ID NO: 53 | BRASSICA NAPUS | SPO11-1 | PN |
| SEQ ID NO: 54 | BRASSICA NAPUS | SPO11-1 | PP |
| SEQ ID NO: 55 | BRASSICA RAPA | SPO11-1 | PN |
| SEQ ID NO: 56 | BRASSICA RAPA | SPO11-1 | PP |
| SEQ ID NO: 57 | GLYCINE MAX | SPO11-1 | PN |
| SEQ ID NO: 58 | GLYCINE MAX | SPO11-1 | PP |
| SEQ ID NO: 59 | GLYCINE MAX | SPO11-1 | PN |
| SEQ ID NO: 60 | GLYCINE MAX | SPO11-1 | PP |
| SEQ ID NO: 61 | GLYCINE MAX | SPO11-1 | PN |
| SEQ ID NO: 62 | GLYCINE MAX | SPO11-1 | PP |
| SEQ ID NO: 63 | MEDICAGO TRUNCATULA | SPO11-1 | PN |
| SEQ ID NO: 64 | MEDICAGO TRUNCATULA | SPO11-1 | PP |
| SEQ ID NO: 65 | ORYZA SATIVA | SPO11-1 | PN |
| SEQ ID NO: 66 | ORYZA SATIVA | SPO11-1 | PP |
| SEQ ID NO: 67 | SETARIA ITALICA | SPO11-1 | PN |
| SEQ ID NO: 68 | SETARIA ITALICA | SPO11-1 | PP |
| SEQ ID NO: 69 | SORGHUM BICOLOR | SPO11-1 | PN |
| SEQ ID NO: 70 | SORGHUM BICOLOR | SPO11-1 | PP |
| SEQ ID NO: 71 | VITIS VINIFERA | SPO11-1 | PN |
| SEQ ID NO: 72 | VITIS VINIFERA | SPO11-1 | PP |
| SEQ ID NO: 73 | ZEA MAYS | SPO11-1 | PN |
| SEQ ID NO: 74 | ZEA MAYS | SPO11-1 | PP |
| SEQ ID NO: 75 | BRASSICA NAPUS | REC8 | PN |
| SEQ ID NO: 76 | BRASSICA RAPA | REC8 | PN |
| SEQ ID NO: 77 | BRASSICA RAPA | REC8 | PP |
| SEQ ID NO: 78 | GLYCINE MAX | REC8 | PN |
| SEQ ID NO: 79 | GLYCINE MAX | REC8 | PP |
| SEQ ID NO: 80 | GLYCINE MAX | REC8 | PN |
| SEQ ID NO: 81 | GLYCINE MAX | REC8 | PP |
| SEQ ID NO: 82 | MEDICAGO TRUNCATULA | REC8 | PN |
| SEQ ID NO: 83 | MEDICAGO TRUNCATULA | REC8 | PP |
| SEQ ID NO: 84 | MEDICAGO TRUNCATULA | REC8 | PN |
| SEQ ID NO: 85 | MEDICAGO TRUNCATULA | REC8 | PP |
| SEQ ID NO: 86 | ORYZA SATIVA | REC8 | PN |
| SEQ ID NO: 87 | ORYZA SATIVA | REC8 | PP |
| SEQ ID NO: 88 | SETARIA ITALICA | REC8 | PN |
| SEQ ID NO: 89 | SETARIA ITALICA | REC8 | PP |
| SEQ ID NO: 90 | SORGHUM BICOLOR | REC8 | PN |
| SEQ ID NO: 91 | SORGHUM BICOLOR | REC8 | PP |
| SEQ ID NO: 92 | VITIS VINIFERA | REC8 | PN |
| SEQ ID NO: 93 | VITIS VINIFERA | REC8 | PP |
| SEQ ID NO: 94 | ZEA MAYS | REC8 | PN |
| SEQ ID NO: 95 | ZEA MAYS | REC8 | PP |
| SEQ ID NO: 96 | BRASSICA NAPUS | CENP-C | PN |
| SEQ ID NO: 97 | BRASSICA NAPUS | CENP-C | PP |
| SEQ ID NO: 98 | BRASSICA NAPUS | CENP-C | PN |
| SEQ ID NO: 99 | BRASSICA NAPUS | CENP-C | PP |
| SEQ ID NO: 100 | BRASSICA RAPA | CENP-C | PN |
| SEQ ID NO: 101 | BRASSICA RAPA | CENP-C | PP |
| SEQ ID NO: 102 | BRASSICA RAPA | CENP-C | PN |
| SEQ ID NO: 103 | GLYCINE MAX | CENP-C | PN |
| SEQ ID NO: 104 | GLYCINE MAX | CENP-C | PP |
| SEQ ID NO: 105 | GLYCINE MAX | CENP-C | PN |
| SEQ ID NO: 106 | GLYCINE MAX | CENP-C | PP |
| SEQ ID NO: 107 | MEDICAGO TRUNCATULA | CENP-C | PN |
| SEQ ID NO: 108 | MEDICAGO TRUNCATULA | CENP-C | PP |
| SEQ ID NO: 109 | ORYZA SATIVA | CENP-C | PN |
| SEQ ID NO: 110 | ORYZA SATIVA | CENP-C | PP |
| SEQ ID NO: 111 | SETARIA ITALICA | CENP-C | PN |
| SEQ ID NO: 112 | SETARIA ITALICA | CENP-C | PP |
| SEQ ID NO: 113 | SORGHUM BICOLOR | CENP-C | PN |
| SEQ ID NO: 114 | SORGHUM BICOLOR | CENP-C | PP |
| SEQ ID NO: 115 | ZEA MAYS | CENP-C | PN |
| SEQ ID NO: 116 | ZEA MAYS | CENP-C | PP |
| SEQ ID NO: 117 | ZEA MAYS | CENP-C | PN |
| SEQ ID NO: 118 | ZEA MAYS | CENP-C | PP |
| SEQ ID NO: 119 | ZEA MAYS | CENP-C | PN |
| SEQ ID NO: 120 | ZEA MAYS | CENP-C | PP |
| SEQ ID NO: 121 | BRASSICA NAPUS | MIS12 | PN |
| SEQ ID NO: 122 | BRASSICA NAPUS | MIS12 | PN |
| SEQ ID NO: 123 | BRASSICA NAPUS | MIS12 | PP |
| SEQ ID NO: 124 | BRASSICA RAPA | MIS12 | PN |
| SEQ ID NO: 125 | BRASSICA RAPA | MIS12 | PP |
| SEQ ID NO: 126 | GLYCINE MAX | MIS12 | PN |
| SEQ ID NO: 127 | GLYCINE MAX | MIS12 | PP |
| SEQ ID NO: 128 | GLYCINE MA | MIS12 | PN |
| SEQ ID NO: 129 | GLYCINE MAX | MIS12 | PP |
| SEQ ID NO: 130 | MEDICAGO TRUNCATULA | MIS12 | PN |
| SEQ ID NO: 131 | MEDICAGO TRUNCATULA | MIS12 | PP |
| SEQ ID NO: 132 | MEDICAGO TRUNCATULA | MIS12 | PN |
| SEQ ID NO: 133 | MEDICAGO TRUNCATULA | MIS12 | PP |
| SEQ ID NO: 134 | ORYZA SATIVA | MIS12 | PN |
| SEQ ID NO: 135 | ORYZA SATIVA | MIS12 | PP |
| SEQ ID NO: 136 | SORGHUM BICOLOR | MIS12 | PN |
| SEQ ID NO: 137 | SORGHUM BICOLOR | MIS12 | PP |
| SEQ ID NO: 138 | VITIS VINIFERA | MIS12 | PN |
| SEQ ID NO: 139 | VITIS VINIFERA | MIS12 | PP |
| SEQ ID NO: 140 | ZEA MAYS | MIS12 | PN |
| SEQ ID NO: 141 | ZEA MAYS | MIS12 | PP |
| SEQ ID NO: 142 | ZEA MAYS | MIS12 | PN |
| SEQ ID NO: 143 | ZEA MAYS | MIS12 | PP |
| SEQ ID NO: 144 | BRASSICA NAPUS | NUF2 | PN |
| SEQ ID NO: 145 | BRASSICA NAPUS | NUF2 | PP |
| SEQ ID NO: 146 | BRASSOCA NAPUS | NUF2 | PN |
| SEQ ID NO: 147 | BRASSICA RAPA | NUF2 | PN |
| SEQ ID NO: 148 | BRASSICA RAPA | NUF2 | PP |
| SEQ ID NO: 149 | GLYCINE MAX | NUF2 | PN |
| SEQ ID NO: 150 | GLYCINE MAX | NUF2 | PP |
| SEQ ID NO: 151 | MEDICAGO TRUNCATULA | NUF2 | PN |
| SEQ ID NO: 152 | MEDICAGO TRUNCATULA | NUF2 | PP |
| SEQ ID NO: 153 | MEDICAGO TRUNCATULA | NUF2 | PN |
| SEQ ID NO: 154 | MEDICAGO TRUNCATULA | NUF2 | PP |
| SEQ ID NO: 155 | ORYZA SATIVA | NUF2 | PN |
| SEQ ID NO: 156 | ORYZA SATIVA | NUF2 | PP |
| SEQ ID NO: 157 | ORYZA SATIVA | NUF2 | PN |
| SEQ ID NO: 158 | ORYZA SATIVA | NUF2 | PP |
| SEQ ID NO: 159 | SETARIA ITALICA | NUF2 | PN |
| SEQ ID NO: 160 | SETARIA ITALICA | NUF2 | PP |
| SEQ ID NO: 161 | SORGHUM BICOLOR | NUF2 | PN |
| SEQ ID NO: 162 | SORGHUM BICOLOR | NUF2 | PP |
| SEQ ID NO: 163 | SORGHUM BICOLOR | NUF2 | PN |
| SEQ ID NO: 164 | SORGHUM BICOLOR | NUF2 | PP |
| SEQ ID NO: 165 | SORGHUM BICOLOR | NUF2 | PN |
| SEQ ID NO: 166 | SORGHUM BICOLOR | NUF2 | PP |
| SEQ ID NO: 167 | VITIS VINIFERA | NUF2 | PN |
| SEQ ID NO: 168 | VITIS VINIFERA | NUF2 | PP |
| SEQ ID NO: 169 | VITIS VINIFERA | NUF2 | PN |
| SEQ ID NO: 170 | VITIS VINIFERA | NUF2 | PP |
| SEQ ID NO: 171 | ZEA MAYS | NUF2 | PN |
| SEQ ID NO: 172 | ZEA MAYS | NUF2 | PP |
| SEQ ID NO: 173 | ZEA MAYS | NUF2 | PN |
| SEQ ID NO: 174 | ZEA MAYS | NUF2 | PP |
| SEQ ID NO: 175 | BRASSICA NAPUS | PRD1 | PN |
| SEQ ID NO: 176 | BRASSICA RAPA | PRD1 | PN |
| SEQ ID NO: 177 | BRASSICA RAPA | PRD1 | PP |
| SEQ ID NO: 178 | GLYCINE MAX | PRD1 | PN |
| SEQ ID NO: 179 | GLYCINE MAX | PRD1 | PP |
| SEQ ID NO: 180 | GLYCINE MAX | PRD1 | PN |
| SEQ ID NO: 181 | GLYCINE MAX | PRD1 | PP |
| SEQ ID NO: 182 | MEDICAGO TRUNCATULA | PRD1 | PN |
| SEQ ID NO: 183 | MEDICAGO TRUNCATULA | PRD1 | PP |
| SEQ ID NO: 184 | ORYZA SATIVA | PRD1 | PN |
| SEQ ID NO: 185 | ORYZA SATIVA | PRD1 | PP |
| SEQ ID NO: 186 | SETARIA ITALICA | PRD1 | PN |
| SEQ ID NO: 187 | SETARIA ITALICA | PRD1 | PP |
| SEQ ID NO: 188 | SORGHUM BICOLOR | PRD1 | PN |
| SEQ ID NO: 189 | SORGHUM BICOLOR | PRD1 | PP |
| SEQ ID NO: 190 | VITIS VINIFERA | PRD1 | PN |
| SEQ ID NO: 191 | VITIS VINIFERA | PRD1 | PP |
| SEQ ID NO: 192 | ZEA MAYS | PRD1 | PN |
| SEQ ID NO: 193 | ZEA MAYS | PRD1 | PP |
| SEQ ID NO: 194 | BRASSICA NAPUS | PRD2 | PN |
| SEQ ID NO: 195 | BRASSICA RAPA | PRD2 | PN |
| SEQ ID NO: 196 | BRASSICA RAPA | PRD2 | PP |
| SEQ ID NO: 197 | BRASSICA RAPA | PRD2 | PN |
| SEQ ID NO: 198 | BRASSICA RAPA | PRD2 | PP |
| SEQ ID NO: 199 | GLYCINE MAX | PRD2 | PN |
| SEQ ID NO: 200 | GLYCINE MAX | PRD2 | PP |
| SEQ ID NO: 201 | GLYCINE MAX | PRD2 | PN |
| SEQ ID NO: 202 | GLYCINE MAX | PRD2 | PP |
| SEQ ID NO: 203 | MEDICAGO TRUNCATULA | PRD2 | PN |
| SEQ ID NO: 204 | MEDICAGO TRUNCATULA | PRD2 | PP |
| SEQ ID NO: 205 | ORYZA SATIVA | PRD2 | PN |
| SEQ ID NO: 206 | ORYZA SATIVA | PRD2 | PP |
| SEQ ID NO: 207 | SETARIA ITALICA | PRD2 | PN |
| SEQ ID NO: 208 | SETARIA ITALICA | PRD2 | PP |
| SEQ ID NO: 209 | SORGHUM BICOLOR | PRD2 | PN |
| SEQ ID NO: 210 | SORGHUM BICOLOR | PRD2 | PP |
| SEQ ID NO: 211 | VITIS VINIFERA | PRD2 | PN |
| SEQ ID NO: 212 | VITIS VINIFERA | PRD2 | PP |
| SEQ ID NO: 213 | ZEA MAYS | PRD2 | PN |
| SEQ ID NO: 214 | :ZEA MAYS | PRD2 | PP |
| SEQ ID NO: 215 | BRASSICA NAPUS | PRD3 | PN |
| SEQ ID NO: 216 | BRASSICA RAPA | PRD3 | PN |
| SEQ ID NO: 217 | BRASSICA RAPA | PRD3 | PP |
| SEQ ID NO: 218 | BRASSICA RAPA | PRD3 | PN |
| SEQ ID NO: 219 | BRASSICA RAPA | PRD3 | PP |
| SEQ ID NO: 220 | GLYCINE MAX | PRD3 | PN |
| SEQ ID NO: 221 | GLYCINE MAX | PRD3 | PP |
| SEQ ID NO: 222 | GLYCINE MAX | PRD3 | PN |
| SEQ ID NO: 223 | GLYCINE MAX | PRD3 | PP |
| SEQ ID NO: 224 | MEDICAGO TRUNCATULA | PRD3 | PN |
| SEQ ID NO: 225 | MEDICAGO TRUNCATULA | PRD3 | PP |
| SEQ ID NO: 226 | ORYZA SATIVA | PRD3 | PN |
| SEQ ID NO: 227 | ORYZA SATIVA | PRD3 | PP |
| SEQ ID NO: 228 | SETARIA ITALICA | PRD3 | PN |
| SEQ ID NO: 229 | SETARIA ITALICA | PRD3 | PP |
| SEQ ID NO: 230 | SORGHUM BICOLOR | PRD3 | PN |
| SEQ ID NO: 231 | SORGHUM BICOLOR | PRD3 | PP |
| SEQ ID NO: 232 | VITIS VINIFERA | PRD3 | PN |
| SEQ ID NO: 233 | VITIS VINIFERA | PRD3 | PP |
| SEQ ID NO: 234 | VITIS VINIFERA | PRD3 | PN |
| SEQ ID NO: 235 | ZEA MAYS | PRD3 | PN |
| SEQ ID NO: 236 | ZEA MAYS | PRD3 | PP |
| SEQ ID NO: 237 | ZEA MAYS | PRD3 | PN |
| SEQ ID NO: 238 | ZEA MAYS | PRD3 | PP |
| SEQ ID NO: 239 | ARABIDOPSIS THALIANA | CENP-O | PN |
| SEQ ID NO: 240 | ARABIDOPSIS THALIANA | CENP-O | PP |
| SEQ ID NO: 241 | BRASSICA NAPUS | CENP-O | PN |
| SEQ ID NO: 242 | BRASSICA RAPA | CENP-O | PN |
| SEQ ID NO: 243 | BRASSICA RAPA | CENP-O | PP |
| SEQ ID NO: 244 | GLYCINE MAX | CENP-O | PN |
| SEQ ID NO: 245 | GLYCINE MAX | CENP-O | PP |
| SEQ ID NO: 246 | GLYCINE MAX | CENP-O | PN |
| SEQ ID NO: 247 | GLYCINE MAX | CENP-O | PP |
| SEQ ID NO: 248 | GLYCINE MAX | CENP-O | PN |
| SEQ ID NO: 249 | GLYCINE MAX | CENP-O | PP |
| SEQ ID NO: 250 | MEDICAGO TRUNCATULA | CENP-O | PN |
| SEQ ID NO: 251 | MEDICAGO TRUNCATULA | CENP-O | PP |
| SEQ ID NO: 252 | ORYZA SATIVA | CENP-O | PN |
| SEQ ID NO: 253 | ORYZA SATIVA | CENP-O | PP |
| SEQ ID NO: 254 | SETARIA ITALICA | CENP-O | PN |
| SEQ ID NO: 255 | SETARIA ITALICA | CENP-O | PP |
| SEQ ID NO: 256 | SETARIA ITALICA | CENP-O | PN |
| SEQ ID NO: 257 | SETARIA ITALICA | CENP-O | PP |
| SEQ ID NO: 258 | SETARIA ITALICA | CENP-O | PN |
| SEQ ID NO: 259 | SETARIA ITALICA | CENP-O | PP |
| SEQ ID NO: 260 | SORGHUM BICOLOR | CENP-O | PN |
| SEQ ID NO: 261 | SORGHUM BICOLOR | CENP-O | PP |
| SEQ ID NO: 262 | SORGHUM BICOLOR | CENP-O | PN |
| SEQ ID NO: 263 | SORGHUM BICOLOR | CENP-O | PN |
| SEQ ID NO: 264 | VITIS VINIFERA | CENP-O | PN |
| SEQ ID NO: 265 | VITIS VINIFERA | CENP-O | PP |
| SEQ ID NO: 266 | ZEA MAYS | CENP-O | PN |
| SEQ ID NO: 267 | ZEA MAYS | CENP-O | PP |
| SEQ ID NO: 268 | ZEA MAYS | CENP-O | PN |
| SEQ ID NO: 269 | ZEA MAYS | CENP-O | PP |
| SEQ ID NO: 270 | ZEA MAYS | CENP-O | PN |
| SEQ ID NO: 271 | ZEA MAYS | CENP-O | PP |
| SEQ ID NO: 272 | GLYCINE MAX | REC8 | PN |
| SEQ ID NO: 273 | GLYCINE MAX | REC8 | PN |
| SEQ ID NO: 274 | GLYCINE MAX | REC8 | PN |
| SEQ ID NO: 275 | ARABIDOPSIS THALIANA | MPRD3 | PN |
| SEQ ID NO: 276 | GLYCINE MAX | MPRD3 GM159 | PN |
| SEQ ID NO: 277 | ARABIDOPSIS THALIANA | MREC8 | PN |
| SEQ ID NO: 278 | GLYCINE MAX | MREC8 GM168C | PN |
| SEQ ID NO: 279 | ARABIDOPSIS THALIANA | MCENH3 A | PN |
| SEQ ID NO: 280 | ARABIDOPSIS THALIANA | 159CENH3 A | PN |
| SEQ ID NO: 281 | ARABIDOPSIS THALIANA | MCENH3 B | PN |
| SEQ ID NO: 282 | ARABIDOPSIS THALIANA | 159CENH3 B | PN |
| SEQ ID NO: 283 | ARABIDOPSIS THALIANA | MCENH3 C | PN |
| SEQ ID NO: 284 | ARABIDOPSIS THALIANA | 159CENH3 C | PN |
| SEQ ID NO: 285 | ARABIDOPSIS THALIANA | MCENH3 D | PN |
| SEQ ID NO: 286 | ARABIDOPSIS THALIANA | 159CENH3 D | PN |
| SEQ ID NO: 287 | ARABIDOPSIS THALIANA | MCENH3 E | PN |
| SEQ ID NO: 288 | ARABIDOPSIS THALIANA | 159CENH3 E | PN |

### I. Apomixis

Apomixis, or asexual reproduction through seed, results in progeny that are genetic clones of the maternal parent. Apomixis requires a non-reduction of the chromosomes from one parental gamete and subsequent parthenogenic development of the embryo. Apomixis may provide a mechanism to maintain heterosis, or hybrid vigor, in crop plants. The present invention involves a combination of two technologies used to produce a self-reproducing hybrid. The first technology is a methodology to produce clonal non-reduction of the genomic content of gametes or mitosis instead of meiosis (MiMe), as demonstrated in Arabidopsis (d′Erfurth, et al., (2009). PLoS Biol 7:e1000124). The second technology has the capacity to induce parent-specific genome elimination at high frequency (CENH3 GFP-tailswap) (Ravi and Chan, (2010) Nature 464:615-618), Genome Elimination induced by a Mix of CENH3 variants (Marimuthu, et al. (2011) Science 331:876). As used herein, “self-reproducing hybrid” refers to hybrid plants capable of perpetuating a heterozygous genome in progeny following self-fertilization. A demonstration of the capacity for these components to produce self-reproducing plants was shown by Marimuthu, et al., (2011) Science 331:876. However, the efficiency of this system is poor and requires significant modifications to become economically and biologically efficient.

A. Mitosis instead of Meiosis

Meiosis is a cell-division mechanism essential for sexually reproducing organisms. In plants, meiosis begins with one diploid cell containing two copies of each chromosome (2n) and produces four haploid gamete cells containing a single recombined copy of each chromosome (1n). Meiosis produces haploid gametes, each having a unique combination of maternal and paternal DNA. Meiosis typically involves chromosomal replication followed by recombination and two rounds of segregation and division. Alternatively, mitosis produces two identical daughter cells following a round of chromosomal replication, segregation, and division.

Inactivation of specific genes controlling meiosis can alter the chromosomal composition of the resultant gametes. For example, a mutation in the dyad gene of Arabidopsis resulted in female meiosis and megasporogenesis producing a dyad of megaspores, rather than a tetrad (Siddiqi, et al., (2000) Arabidopsis Development 127:197-207). By selectively inactivating a combination of meiosis-related genes, the meiotic divisions can be replaced by a mitotic-like division, resulting in unreduced gametes that are identical to the parent cell (d′Erfurth, et al., (2009) PLoS Biol 7(6):e1000124). Inactivating osd1 resulted in an Arabidopsis mutant that did not undergo meiosis II, giving rise to diploid gametes having recombined chromosomes. Further, a double spo11-1/rec8 Arabidopsis mutant avoids the first division of meiosis and, instead, undergoes a mitotic-like division, followed by an unbalanced second division resulting in chromosomally unbalanced and sterile gametes. A triple osd1/spo11-1/rec8 mutant, designated MiMe, led to a mitotic-like first division due to the Atspo11-1 and Atrec8 mutations, and an absent second meiotic division due to the osd1 mutation. Thus, the MiMe mutations resulted in the replacement of meiosis with a mitotic-like division, thereby producing gametes having genetically identical chromosomes as the parent.

Various compositions are provided comprising suppression cassettes encoding inhibitory polynucleotides that decrease the activity of target polypeptides. In particular embodiments, silencing elements are provided encoding inhibitory polynucleotides that decrease the activity of Spo11-1, Rec8 or Osd1. In specific embodiments, silencing elements encoding inhibitory polynucleotides are provided that decrease the activity of Spo11-1, Rec8 and Osd1, thereby producing the MiMe phenotype. Such nucleic acid molecule constructs are referred to herein as “MiMe silencing elements”.

The Spoil family of plant proteins are homologs of archaeal DNA topoisomerase VIA subunit (topo VIA), which participates in DNA replication. Spo11-1 specifically contributes to the creation of double stranded breaks necessary for recombination in the early phases of meiosis, and inactivating Spo11-1 results in sterile plants. Rec8 is responsible for localization of the axial chromosomal elements during meiosis. Following meiosis I, Rec8 has been identified at the centromere, and the depletion of Rec8 eliminated centromeric cohesion. Thus, the presence of Rec8 at the centromere has been thought to maintain sister chromatid cohesion throughout meiosis I (see, Stoop-Myer et al Meiosis: Rec8 is the reason for cohesion (1999) Nat Cell Biol 1:E125-7). Osd1 (omission of second division) is an IV14-like protein identified as a result of its co-regulation with other meiotic genes. In osd1 deficient Arabidopsis plants, the products of male meiosis were dyads instead of tetrads. Further, only tetrapoloid (4n) and triploid (3n) progeny were detected from self-pollinated osd1 deficient mutants. Thus, inactivation of osd1 produced functional diploid gametes due to absence of the second meiotic division.

In particular embodiments of the present invention, suppression cassettes provided elsewhere herein comprise MiMe silencing elements operably linked to promoters that drive expression in a plant. In some embodiments, promoters operably linked to MiMe silencing elements are inducible promoters. For example, in specific embodiments, MiMe silencing elements are operably linked to inducible promoters activated by a transactivator. As discussed elsewhere herein, the transactivator can be provided in the same plant or in a separate plant subsequently crossed with a plant comprising a MiMe silencing element operably linked to a transactivator-inducible promoter, thereby producing functional diploid gametes.

In some embodiments, these or other genes may be targeted through knockout, dominant negative allele expression, as hypomorph, as hypermorph, protein inactivation or through silencing. In some embodiments, Spo11-2, Dfo, Prd1, Prd2, Prd3, or Tam1, genes or any ortholog thereof are targeted. In other embodiments, the targeted genes may be am1, am2, pam1, pam2, as1, dsy1, dy1, st1, el1, dv1, va1, va2, or any ortholog thereof. In yet another embodiment, the targeted genes may be AGO4 (ARGONAUTE 4), AGO6 (ARGONAUTE 6), AGO8 (ARGONAUTE 8), AGO9 (ARGONAUTE 9), CMTS (CHROMOMETHYLASE 3), DCL3 (DICER-LIKE 3), DRM2 (DOMAINS REARRANGED METHYLASE 2), EXS1 (EXTRA SPOROGENOUS CELLS1), IDN2 (INVOLVED IN DE NOVO 2), MET1 (METHYL TRANSFERASE 1), NPRD1a (NUCLEAR POLYMERASE D 1a), NRPD1b (NUCLEAR POLYMERASE D 1b), NRPD2 (NUCLEAR POLYMERASE D2), NRPE1 (NUCLEAR RNA POLYMERASE E 1), NRPE2 (NUCLEAR RNA POLYMERASE E 2), RDR2 (RNA-DEPENDENT RNA POLYMERASE 2), RDR6 (RNA-DEPENDENT RNA POLYMERASE 6), SGS3 (SUPPRESSOR OF GENE SILENCING 3), SUVH2 (SUPPRESSOR OF VARIEGATION 3-9 HOMOLOG 2), and SUVH9 (SUPPRESSOR OF VARIEGATION 3-9 HOMOLOG 9), or any ortholog thereof.

B. Genome Elimination

A method for producing plants that only inherit chromosomes from one parent can significantly accelerate plant breeding by providing plants in a single generation without the need for generations of inbreeding. By altering the structure of histones of the kinetochore complex (centromere-specific polypeptides), such as CENH3, the chromosomes of the altered parent are eliminated in the zygote, thereby creating haploid plants. The resultant haploid plants have very high male sterility, but when pollinated by wild-type males, the female genome is eliminated at the first zygotic mitosis. In addition to near total male sterility, the resultant plants also show very low rates of female fecundity. In some embodiments, active CENH3 mutant expression can be more widely expressed through the ovule, but a egg cell promoter could be used to express a wild-type CENH3 thus “rescuing” the maternal genome in the resulting zygote but leading to male genome elimination in the zygote and thus a maternal clone.

Various compositions that employ wild-type and modified kinetochore (centromere-specific) proteins are provided. Methods and compositions are provided comprising, for example, the CENH3, CENPC, MCM21, MIS12, NDC80 or NUF2 centromere-specific proteins. CENH3 proteins are discussed below. Structural and/or functional features of the other kinetochore proteins have been described in, for example, Du, et al., (2010) PLoS Genet. 6:e1000835; Talbert, et al., (2004) J. Biol. 3:18; Sato, et al., (2005) Chrom. Res. 13:827-834; Pidoux, et al., (2000) Opin. Cell Biol. 12:308-319; Du, et al., (2007) Chrom. Res. 15:767-775; Zhang and Dawe, (2011) Chrom. Res. (Mar. 19, 2011 epub) 1-10 and Meraldi, et al., (2006) Genome Biol. 7:R23; all of which are herein incorporated by reference.

In particular, various compositions that employ CENH3 and modified variants thereof are provided. CENH3 proteins are a well-characterized class of H3 histone protein variants associated with centromere function and development as one of the proteins that form the kinetochore complex. CENH3 proteins are characterized by a variable tail domain, which does not form a rigid secondary structure, and a conserved histone fold domain made up of three α-helical regions connected by loop sections. Additional structural and functional features of CENH3 proteins can be found in, e.g., Cooper, et al., (2004) Mol Biol Evol. 21(9):1712-8, Malik, et al., (2003) Nat Struct Biol. 10(11):882-91; Black, et al., (2008) Curr Opin Cell Biol. 20(1):91-100.

The CENH3 histone fold domain is conserved between CENH3 proteins from different species and can be distinguished by three a-helical regions connected by loop sections. While it will be appreciated that the exact location of the histone fold domain will vary in CENH3 variants, it will be found at the carboxyl terminus of an endogenous (wild-type) CENH3 protein. The border between the tail domain and the histone fold domain of CENH3 proteins is at, within, or near (i.e., within 5, 10, 15, 20 or 25 amino acids from the “P” of) the conserved PGTVAL (SEQ ID NO: 1) sequence. The PGTVAL sequence is approximately 81 amino acids from the N terminus of the Arabidopsis CENH3 protein, though the distance from the N terminus of different endogenous CENH3 proteins varies. Thus, in some embodiments, the histone fold region of CENH3 employed in the tailswap proteins includes all of the C-terminal amino acids of an endogenous CENH3 protein (or a protein substantially similar to the endogenous sequence) up to and including the PGTVAL. In other embodiments, the tailswap proteins can comprise more or less of the CENH3 sequence. For example, in some embodiments, the tailswap will comprise the C-terminal sequence of a CENH3 protein, but only up to an amino acid 5, 10, 15, 20 or 25 amino acids in the C-terminal direction from the “P” of the conserved PGTVAL sequence. In some embodiments, the tailswap will comprise the C-terminal sequence of a CENH3 protein, but only up to 5, 10, 15, 20 or 25 amino acids in the N-terminal direction from the “P” of the conserved PGTVAL sequence.

Any number of mutations of CENH3 can be introduced into a CENH3 protein to generate a mutated (including but not limited to a recombinantly altered) CENH3 protein capable of generating haploid plants when expressed in a plant having suppressed expression of an endogenous CENH3 protein, and wherein wild-type CENH3 protein is provided to the resulting transgenic plant. For example, wild-type CENH3 can be provided by crossing a transgenic plant expressing an active CENH3 mutant to a plant expressing a wild-type CENH3 protein. Active CENH3 mutant proteins can be identified, for example, by random mutagenesis, by single or multiple amino acid targeted mutagenesis, by generation of complete or partial protein domain deletions, by fusion with heterologous amino acid sequences, or by combinations thereof. Active centromere-specific mutant polypeptides refer to polypeptides that, when expressed in a plant in which the wild-type centromere-specific polypeptide is knocked out or inactivated, result in viable plants, which viable plants when crossed to a wild-type plant, produce haploid progeny at a more than normal frequency (e.g., at least 0.1, 0.5, 1, 5, 10, 20% or more). For example, “active CENH3 mutant proteins” refer to proteins that, when expressed in a plant in which CENH3 is knocked out or inactivated, result in viable plants, which viable plants when crossed to a wild-type plant, produce haploid progeny at a more than normal frequency (e.g., at least 0.1, 0.5, 1, 5, 10, 20% or more). Active mutated CENH3 proteins can be readily tested by recombinant expression of the mutated CENH3 protein in a plant lacking endogenous CENH3 protein, crossing the transgenic plant (as a male or female, depending on fertility) to a plant expressing wild-type CENH3 protein, and then screening for the production of haploid progeny.

In some embodiments, an active CENH3 mutant protein is identical to an endogenous CENH3 protein but for 1, 2, 3, 4, 5, 6, 7, 8 or more (e.g., 1-2, 1-4, 1-8) amino acids. For example, in some embodiments, the endogenous wild-type protein from the plant is identical or substantially identical to SEQ ID NO: 5 and the active CENH3 mutant protein differs from the endogenous CENH3 protein by 1, 2, 3, 4, 5, 6, 7, 8 or more (e.g., 1-2, 1-4, 1-8) amino acids. It is believed that active CENH3 mutants include, for example, proteins comprising: a heterologous amino acid sequence (including but not limited to green fluorescent protein (GFP)) linked to a CENH3 truncated or complete tail domain or non-CENH3 tail domain, either of which is linked to a CENH3 histone fold domain or a CENH3 truncated tail domain, the heterologous CENH3 tail domain or non-CENH3 tail domain, either of which is linked to a CENH3 histone fold domain. In some embodiments, the active CENH3 mutant protein comprises a fusion of an amino-terminal heterologous amino acid sequence to the histone-fold domain of a CENH3 protein. Generally, the histone fold domain will be identical or at least substantially identical to the CENH3 protein endogenous to the organism in which the active CENH3 mutant protein will be expressed. In some embodiments, the active CENH3 mutant protein will include a histone tail domain, which can be, for example, a non-CENH3 tail domain, or a CENH3 tail domain.

It is believed that a large number of different amino acid sequences, when linked to a protein comprising a CENH3 histone-fold domain and a sequence that can function as or replace a histone tail domain, can be used to construct an active CENH3 mutant. In some embodiments, a heterologous sequence is linked directly to the CENH3 histone-fold domain.

In some embodiments, the heterologous sequence is an intervening amino acid sequence linked to the CENH3 histone-fold domain. In some embodiments, the intervening amino acid sequence is an intact or truncated CENH3 tail domain. The heterologous amino acid sequence, in combination with the histone-fold domain, will be sufficient to prevent the lethality associated with loss of endogenous CENH3, but will sufficiently disrupt centromeres to allow for production of haploid progeny, as discussed herein. Thus, in some embodiments, the heterologous amino acid sequence will comprise a portion that is, or mimics the function of, a histone tail domain and optionally can also comprise a bulky amino acid sequence that disrupts centromere function. In certain embodiments, at least a portion of the heterologous amino acid sequence of the mutated CENH3 protein comprises any amino acid sequence of at least 10, 20, 30, 40, 50, e.g., 10-30, 10-50, 20-50, 30-50 amino acids, optionally lacking a stable secondary structure (e.g., lacking coils, helices or beta-sheets). In some embodiments, the tail domain has less than 90, 80 or 70% identity with the tail domain (e.g., the N-terminal 135 amino acids) of the CENH3 protein endogenous to the organism in which the mutated CENH3 protein will be expressed. In some embodiments, the tail domain of the mutated CENH3 protein comprises the tail domain of a non-CENH3 histone protein, including but not limited to an H3 histone protein. In some embodiments, the tail domain of the mutated CENH3 protein comprises the tail domain of a non-CENH3 histone protein endogenous to the organism in which the mutated CENH3 protein will be expressed. In some embodiments, the tail domain of the mutated CENH3 protein comprises the tail domain of a homologous or orthologous (from a different plant species) CENH3 tail. For example, it has been found that GFP fused to a maize CENH3 tail domain linked to an Arabidopsis CENH3 histone-fold domain is active.

As noted above, in some embodiments, the tail domain of an H3 histone (not to be confused with a CENH3 histone) is used as the tail domain portion of the active CENH3 mutant protein (these embodiments are sometimes referred to as “tailswap” proteins). Plant H3 tail domains are well conserved in various organisms.

In some embodiments, active CENH3 mutant proteins will lack at least a portion (e.g., at least 5, 10, 15, 20, 25, 30 or more amino acids) of the endogenous CENH3 N-terminal region, and thus, in some embodiments, will have a truncated CENH3 tail domain compared to a wild-type endogenous CENH3 protein. Active CENH3 mutant proteins may, or may not, be linked to a heterologous sequence.

Optionally, the heterologous amino acid sequence can comprise, or further comprise, one or more amino acid sequences at the amino and/or carboxyl terminus and/or linking the tail and histone fold domains. For example, in some embodiments, the active CENH3 mutant protein (e.g., a tailswap or other active CENH3 mutant protein) comprises a heterologous amino acid sequence linked to the amino end of the tail domain. In some embodiments, the heterologous sequence is linked to the amino terminus of an otherwise wild-type CENH3 protein, wherein the heterologous sequence interferes with centromere function. For example, it has been found that GFP, when linked to wild-type CENH3, sufficiently disrupts centromeres to allow for production of haploid progeny. It is believed that the heterologous sequence can be any sequence that disrupts the CENH3 protein's ability to maintain centromere function. Thus, in some embodiments, the heterologous sequence comprises an amino acid sequence of at least 5, 10, 15, 20, 25, 30, 50 or more kD.

In some embodiments, the active CENH3 mutant protein will comprise a protein domain that acts as a detectable or selectable marker. For example, an exemplary selectable marker protein is fluorescent or an antibiotic or herbicide resistance gene product. Selectable or detectable protein domains are useful for monitoring the presence or absence of the mutated CENH3 protein in an organism.

In other embodiments, expression cassettes are provided comprising an active CENH3 mutant protein operably linked to a promoter that drives expression in a plant. In particular embodiments, promoters operably linked to active CENH3 mutant proteins are inducible promoters or tissue-specific promoters. For example, in specific embodiments, active CENH3 mutant proteins are operably linked to promoters specifically induced in the ovule of a plant.

In some embodiments, expression cassettes comprising a nucleotide sequence encoding wild-type CENH3 operably linked to a promoter that drives expression in a plant are provided. In particular embodiments, promoters operably linked to nucleotide sequences encoding wild-type CENH3 are tissue specific promoters. For example, nucleotide sequences encoding wild-type CENH3 operably linked to central cell-specific promoters (e.g., AT-DD65 promoter, AT-DD9 promoter, or AT-DD25 promoter) that drive expression of wild-type CENH3 in the central cell of a plant are provided. Expression cassettes comprising a central-cell specific promoter operably linked to a polynucleotide encoding wild-type CENH3 can be provided in the same parental plant as CENH3 suppression cassettes and/or the same parental plant as active CENH3 mutant expression cassettes.

Further provided are inhibitory polynucleotides that decrease the activity of wild-type CENH3. In some embodiments, suppression cassettes comprising a silencing element encoding inhibitory polynucleotides that decrease the activity of wild-type CENH3 operably linked to an inducible promoter that drives expression in a plant are provided. In specific embodiments, suppression cassettes comprising a silencing element encoding inhibitory polynucleotides that decrease the activity of wild-type CENH3 operably linked to a promoter specifically induced by a transactivator are provided. As discussed elsewhere herein, the transactivator can be provided in the same plant or in a separate plant subsequently crossed with a plant comprising a CENH3 silencing element operably linked to a transactivator-inducible promoter, thereby activating the CENH3 silencing element in the progeny plant. In some embodiments, a recombinase may be used to eliminate a buffering component between a promoter and the DNA region encoding the inhibitory polynucleotides.

In a particular embodiment, a first plant comprising an active CENH3 mutant expression cassette comprising a central cell-specific promoter, a CENH3 suppression cassette comprising a transactivator A-inducible promoter, a CENH3 expression cassette comprising an egg-cell specific promoter and a transactivator B expression cassette comprising an ovule-specific promoter is crossed with a second plant comprising an active CENH3 mutant expression cassette comprising a sperm-cell preferred promoter, a MiMe suppression cassette comprising a transactivator B-inducible promoter and a transactivator A expression cassette comprising an ovule-specific promoter, producing a tetraploid zygote that subsequently loses the male genome from the sperm cell following a generation of self fertilization, ultimately resulting in a self-reproducing hybrid progeny plant.

C. Methods for Producing Self-Reproducing Hybrid Plants

A single-cross hybrid plant results from the cross of two inbred varieties, each of which has a genotype that complements the genotype of the other. A hybrid progeny of the first generation is designated F1. In the development of commercial hybrids in a plant breeding program, the F1 hybrid plants are most desired. F1 hybrids are more vigorous than their inbred parents. This hybrid vigor, or heterosis, can be manifested in many polygenic traits, including increased vegetative growth and increased yield.

Crossing a pollen parent plant comprising cassettes for suppressing the activity of an endogenous kinetochore complex protein (e.g., CENH3, CENPC, MCM21, MIS12, NDC80 or NUF2 protein) in progeny ovules and cassettes for expressing an endogenous kinetochore complex protein in the egg cell of progeny to an ovule parent plant comprising cassettes for expressing inhibitory polynucleotides resulting in a MiMe phenotype in progeny and cassettes for expressing an active mutated kinetochore complex protein (e.g., a tailswap or other mutated CENH3 or non-CENH3 kinetochore complex protein) in the ovule and sperm-cells of progeny as described herein, will result in at least some progeny (e.g., at least 0.1%, 0.5%, 1%, 5%, 10%, 20% or more) that are diploid following self-fertilization, and comprise only chromosomes from the female parent that expresses the kinetochore complex protein. Thus, the present invention allows for the generation of clonal diploid plants capable of self-reproducing.

While the present invention is not known to depend on a particular mechanism, it is believed that the methods of the present invention increase self-reproducing hybrid seed viability by preventing parental genome elimination in the central cell of the ovule. It is further believed that complementing the central cell with active mutant CENH3, such as that delivered from the sperm cells, allows proper endosperm development by maintaining a 2M:1P (2 maternal:1 paternal) ratio necessary for proper endosperm development.

In some embodiments, a method for producing a self-reproducing hybrid plant is provided comprising crossing a first plant comprising a first suppression cassette comprising a MiMe silencing element and a first expression cassette expressing an active CENH3 mutant protein with a second plant comprising a second suppression cassette that reduces the level of wild-type CENH3 and a second expression cassette expressing CENH3 specifically in the egg cell. Self fertilization of the resultant progeny plant results in the elimination of the male diploid genome in the zygote and normal development of the endosperm, thereby producing a self-reproducing hybrid plant.

### II. Compositions

Compositions disclosed herein provide nucleic acid molecule constructs comprising expression and suppression cassettes comprising polynucleotides related to meiosis or genome elimination. As used herein, “meiosis-related” or “MiMe-related” refers to those polynucleotides encoding polypeptides involved directly or indirectly in the process of meiosis. Further, as used herein, “kinetochore” or “CENH3” refers to the specialized protein structure on choromosomes that mediates the attachment of spindle fibers during cell division.

Decreasing the level of polynucleotides encoding such polypeptides or decreasing the activity of the encoded polypeptides could result in absence of the first meiotic division, meiosis II, or unbalanced second meiotic divisions. Methods for measuring the level of polynucleotides and activity of the encoded polypeptides are disclosed elsewhere herein. For example, RNA transcripts are monitored through the use of qRT-PCR. SybrGreen or TaqMan probes may be used. Polypeptide activities are assayed indirectly through cytogenetics and progeny segregation analysis.

By “reduces”, “reducing”, “decrease”, or “decreasing” the expression level of a polynucleotide or activity of a polypeptide encoded thereby is intended to mean, the polynucleotide or polypeptide level of the target sequence is statistically lower than the polynucleotide level or polypeptide level of the same target sequence in an appropriate control plant that is not expressing the silencing element. In particular embodiments of the invention, reducing the polynucleotide level and/or the polypeptide level of the target sequence in a plant according to the invention results in less than 95%, less than 90%, less than 80%, less than 70%, less than 60%, less than 50%, less than 40%, less than 30%, less than 20%, less than 10% or less than 5% of the polynucleotide level or the level of the polypeptide encoded thereby, of the same target sequence in an appropriate control plant. Methods to assay for the level of the RNA transcript, the level of the encoded polypeptide or the activity of the polynucleotide or polypeptide are known in the art and discussed elsewhere herein.

A. Silencing Elements

Further provided are nucleic acid molecules comprising nucleotide sequences encoding inhibitory nucleic acids, and fragments and variants thereof that are useful in decreasing the level of proteins responsible for normal meiosis and wild-type kinetochore activity. Such fragments and variants are useful in silencing elements and suppression cassettes.

By “silencing elements” is intended polynucleotides that can reduce or eliminate the expression level of a target sequence by influencing the level of the target RNA transcript or, alternatively, by influencing translation and thereby affecting the level of the encoded polypeptide. As used herein, a “target sequence” or “target polynucleotide” comprises any sequence that one desires to reduce the level of expression. In specific embodiments, the target sequence comprises the nucleotide sequence set forth in SEQ ID NO: 2, 3 and 4 and decreasing the level of expression of the target sequence results in an alteration of normal meiosis activity. In other embodiments, the target sequence comprises the nucleotide sequence set forth in SEQ ID NO: 5. Methods to assay for functional silencing elements that are capable of reducing or eliminating the level of a sequence of interest are known in the art.

As discussed in further detail below, silencing elements can include, but are not limited to, a sense suppression element, an antisense suppression element, a double stranded RNA, an siRNA, an amiRNA, an miRNA or a hairpin suppression element. Non-limiting examples of silencing elements that can be employed to decreased expression of meiosis-related genes or CENH3 genes comprise fragments and variants of the sense or antisense sequence of the sequences set forth in SEQ ID NOS: 2, 3, 4 and/or 5. In other embodiments, dominant negative mutants, directed mutation or protein fragments may be used to suppress, or alter target function.

i. Sense Suppression Elements

Silencing elements of the invention may comprise a sense suppression element. As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide or all or part of both the coding sequence and the untranslated regions of the target polynucleotide.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323, herein incorporated by reference. The sense suppression element can be any length so long as it allows for the suppression of the targeted sequence. The sense suppression element can be, for example, the full-length nucleotide sequence of SEQ ID NOS: 2, 3, 4 and 5 or about 10, 15, 16, 17, 18, 19, 20, 22, 25, 30, 50, 100, 150, 200, 250, 300, 350, 400, 450, 500 nucleotides or longer of the nucleotides set forth in SEQ ID NOS: 2, 3, 4 and 5. In other embodiments, the sense suppression element can be, for example, the full-length nucleotide sequence of SEQ ID NOS: 2, 3, 4 and 5 or about 10, 15, 16, 17, 18, 19, 20, 22, 25, 30, 50, 100, 150, 200, 250, 300, 350, 400, 450, 500, 600, 700, 900, 1000, 1100, 1200, 1300, 1400, 1500 nucleotides or longer of the nucleotides set forth in SEQ ID NOS: 2, 3, 4 and 5.

ii. Antisense Suppression Elements

Silencing elements of the invention may comprise an antisense suppression element. As used herein, an “antisense suppression element” comprises a polynucleotide that is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide.

In specific embodiments, the antisense suppression element comprises at least 85%, 90%, 91%, 92%, 93%, 94%, 95%, 96%, 97%, 98% or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide.

In one example, sequences of at least about 15, 16, 17, 18, 19, 20, 22, 25, 50, 100, 200, 300, 400, 450, 500 nucleotides or longer of the nucleotides set forth in SEQ ID NOS: 2, 3, 4 and 5, or a complement thereof, may be used. In another example, sequences of at least about 15, 16, 17, 18, 19, 20, 22, 25, 50, 100, 200, 300, 400, 450, 500, 600, 700, 900, 1000, 1100, 1200, 1300, 1400, 1500 nucleotides or longer of the nucleotides set forth in SEQ ID NOS: 2, 3, 4 and 5, or a complement thereof, may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference.

iii. Double Stranded RNA Suppression Element

Silencing elements of the invention may comprise a double stranded RNA silencing element. A “double stranded RNA silencing element” or “dsRNA” comprises at least one transcript that is capable of forming a dsRNA. Thus, a “dsRNA silencing element” includes a dsRNA, a transcript or polyribonucleotide capable of forming a dsRNA, or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of least two distinct RNA strands. The dsRNA molecule(s) employed in the methods and compositions of the invention mediate the reduction of expression of a target sequence, for example, by mediating RNA interference “RNAi” or gene silencing in a sequence-specific manner. In the context of the present invention, the dsRNA is capable of reducing or eliminating the level or expression of a target polynucleotide or the polypeptide encoded thereby in a plant.

The dsRNA can reduce or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel, et al., (2004) Science 303:672-676; Pal-Bhadra, et al., (2004) Science 303:669-672; Allshire, (2002) Science 297:1818-1819; Volpe, et al., (2002) Science 297:1833-1837; Jenuwein, (2002) Science 297:2215-2218 and Hall, et al., (2002) Science 297:2232-2237. Methods to assay for functional dsRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (sRNA), double-stranded RNA (dsRNA), micro-RNA (miRNA), artificial micro-RNA (amiRNA), hairpin RNA, short hairpin RNA (shRNA), post-transcriptional gene silencing RNA (ptgsRNA), and others.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand” and the strand homologous to the target polynucleotide is the “sense strand.”

In another embodiment, the dsRNA comprises a hairpin RNA. A hairpin RNA comprises an RNA molecule that is capable of folding back onto itself to form a double-stranded structure. Multiple structures can be employed as hairpin elements. In specific embodiments, the dsRNA suppression element comprises a hairpin element that comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., and 3 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication Number WO 2002/00904, herein incorporated by reference. In specific embodiments, the loop region can be optimized to be as short as possible while still providing enough intramolecular flexibility to allow the formation of the base-paired stem region. Accordingly, the loop sequence is generally less than about 1500, 1400, 1300, 1200, 1100, 1000, 900, 800, 700, 600, 500, 400, 300, 200, 100, 50, 25, 20, 19, 18, 17, 16, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

In specific embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000 or more than 1000 nucleotides that share sufficient sequence identity to the target polynucleotide to allow for a decrease in expression levels of the target polynucleotide when expressed in an appropriate cell.

In further embodiments, the domain of the first, the second, and/or the third segment has 100% sequence identity to the target polynucleotide. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99% or greater sequence identity to a region of the target polynucleotide. The sequence identity of the domains of the first, the second and/or the third segments to the target polynucleotide need only be sufficient to decrease expression of the target polynucleotide of interest. See, for example, Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hairpin RNA constructs to silence gene expression in vivo has been described by Panstruga, et al., (2003) Mol. Biol. Rep. 30:135-140, herein incorporated by reference.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s) and any combination thereof. In some instances, to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers, et al., (2003) J. Biol. Chem 278:7108-7118 and Yang, et al., (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

In particular embodiments, the hairpin RNAs of the invention may also comprise an intron. For such intron-containing hairpin RNAs, the interfering molecules have the same general structure as for the hairpin RNAs described herein above, but the RNA molecule additionally comprises an intron that is capable of being spliced in the cell in which the hairpin RNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using intron-containing hairpin RNA-mediated interference. Methods for using intron-containing hairpin RNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295, and US Patent Application Publication Number 2003/0180945, each of which is herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz, et al., (2002) PNAS 99(4):16499-16506 and Mette, et al., (2000) EMBO J 19(19):5194-5201.

In other embodiments, the dsRNA can comprise a small RNA (sRNA). sRNAs can comprise both micro RNA (miRNA) and short-interfering RNA (siRNA) (Meister and Tuschl, (2004) Nature 431:343-349 and Bonetta, et al., (2004) Nature Methods 1:79-86). miRNAs are regulatory agents comprising about 19 ribonucleotides which are highly efficient at inhibiting the expression of target polynucleotides. See, for example Javier, et al., (2003) Nature 425:257-263, herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

When expressing an miRNA, it is recognized that various forms of an miRNA can be transcribed including, for example, the primary transcript (termed the “pri-miRNA”) which is processed through various nucleolytic steps to a shorter precursor miRNA (termed the “pre-miRNA”), the pre-miRNA or the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto, et al., (2004) Genes & Development 18:2237-2242 and Guo, et al., (2005) Plant Cell 17:1376-1386).

Artificial microRNAs (amiRNAs) have recently been described in Arabidopsis targeting viral mRNA sequences (Niu, et al., (2006) Nature Biotechnology 24:1420-1428) or endogenous genes (Schwab, et al., (2006) Plant Cell 18:1121-1133). The amiRNA construct can be expressed under different promoters in order to change the spatial pattern of silencing (Schwab, et al., (2006) Plant Cell 18:1121-1133). Artificial miRNAs replace the microRNA and its complementary star sequence in a precursor miRNA and substitute sequences that target an mRNA to be silenced. Silencing by endogenous miRNAs can be found in a variety of spatial, temporal, and developmental expression patterns (Parizotto, et al., (2007) Genes Dev 18:2237-2242; Alvarez, et al., (2006) Plant Cell 18:1134-51). Artificial miRNA can be constructed to both capture and extend the diversity and specificity in the patterns of silencing.

The methods and compositions of the invention can employ silencing elements that, when transcribed, form a dsRNA molecule. Accordingly, the heterologous polynucleotide being expressed need not form the dsRNA by itself, but can interact with other sequences in the plant cell to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, US Patent Application Publication Number 2007/0130653, entitled “Methods and Compositions for Gene Silencing”, herein incorporated by reference. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

In specific embodiments, the compositions of the invention include nucleic acid molecules that comprise the nucleotide sequence of Spo11-1 (SEQ ID NO: 2), Osd1 (SEQ ID NO: 3), Rec8 (SEQ ID NO: 4) and CENH3 (SEQ ID NO: 5) nucleotide sequences. Alternatively, such nucleic acid molecules comprise a nucleotide sequence that selectively hybridizes with SEQ ID NOS: 2, 3, 4 and/or 5. Furthermore, such isolated polynucleotides may comprise a nucleotide sequence comprising the complementary sequence to SEQ ID NOS: 2, 3, 4 and/or 5 or the complementary sequence to a nucleotide sequence that selectively hybridizes with SEQ ID NOS: 2, 3, 4 and/or 5.

iv. Gene mutation and homologous recombination

Guide RNA/CAS Endonuclease Systems
(1) CRISPR loci

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times-also referred to as CRISPR-repeats) which are partially palindromic. The repeated sequences (usually specific to a species) are interspaced by variable sequences of constant length (typically 20 to 58 by depending on the CRISPR locus (International Patent Application Publication Number WO 2007/024097, published Mar. 1, 2007).

CRISPR loci were first recognized in E. coli (Ishino, et al., (1987) J. Bacterial. 169:5429-5433; Nakata, et al., (1989) J. Bacterial. 171:3553-3556). Similar interspersed short sequence repeats have been identified in Haloferax mediterranei, Streptococcus pyogenes, Anabaena and Mycobacterium tuberculosis (Groenen, et al., (1993) Mol. Microbiol. 10:1057-1065; Hoe, et al., (1999) Emerg. Infect. Dis. 5:254-263; Masepohl, et al., (1996) Biochim. Biophys. Acta 1307:26-30; Mojica, et al., (1995) Mol. Microbiol. 17:85-93). The CRISPR loci differ from other SSRs by the structure of the repeats, which have been termed short regularly spaced repeats (SRSRs) (Janssen, et al., (2002) OMICS J. Integ. Biol. 6:23-33; Mojica, et al., (2000) Mol. Microbiol. 36:244-246). The repeats are short elements that occur in clusters, that are always regularly spaced by variable sequences of constant length (Mojica, et al., (2000) Mol. Microbiol. 36:244-246).

(2) Cas Genes, Cas Endonucleases

As used herein, the term “Cas gene” refers to a gene that is generally coupled, associated or close to or in the vicinity of flanking CRISPR loci. The terms “Cas gene”, “CRISPR-associated (Cas) gene” are used interchangeably herein. A comprehensive review of the Cas protein family is presented in Haft, et al., (2005) Computational Biology, PLoS Comput Biol 1(6):e60. doi:10.1371/journal.pcbi.0010060.

As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

As used herein, the term “Cas endonuclease” refers to a Cas protein encoded by a Cas gene, wherein said Cas protein is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence

In one embodiment, the Cas endonuclease gene is a Cas9 endonuclease, such as but not limited to, Cas9 genes listed in SEQ ID NOS: 462, 474, 489, 494, 499, 505 and 518 of International Patent Application Number WO 2007/024097, published Mar. 1, 2007, and incorporated herein by reference. In another embodiment, the Cas endonuclease gene is plant, maize or soybean optimized Cas9 endonuclease. In another embodiment, the Cas endonuclease gene is operably linked to a SV40 nuclear targeting signal upstream of the Cas codon region and a bipartite VirD2 nuclear localization signal (Tinland, et al., (1992) Proc. Natl. Acad. Sci. USA 89:7442-6) downstream of the Cas codon region.

In one embodiment, the Cas endonuclease gene is a plant codon optimized streptococcus pyogenes Cas9 gene that can recognize any genomic sequence of the form N(12-30)NGG can in principle be targeted.

Endonucleases are enzymes that cleave the phosphodiester bond within a polynucleotide chain, and include restriction endonucleases that cleave DNA at specific sites without damaging the bases. Restriction endonucleases include Type I, Type II, Type III and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 by or more (International Patent Application Number PCT/US12/30061 filed on Mar. 22, 2012). Meganucleases have been classified into four families based on conserved sequence motifs, the families are the LAGLIDADG, GIY-YIG, H-N-H and His-Cys box families. These motifs participate in the coordination of metal ions and hydrolysis of phosphodiester bonds. HEases are notable for their long recognition sites and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer, (1994) Curr Op Biotechnol 5:521-7 and Sadowski, (1993) FASEB 7:760-7. In some examples the recombinase is from the Integrase or Resolvase families.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, Fokl. The unique, modular TAL effector DNA binding domain allows for the design of proteins with potentially any given DNA recognition specificity (Miller, et al., (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three or four zinc fingers, for example having a C2H2 structure, however other zinc finger structures are known and have been engineered. Zinc finger domains are amenable for designing polypeptides which specifically bind a selected polynucleotide recognition sequence. ZFNs consist of an engineered DNA-binding zinc finger domain linked to a non-specific endonuclease domain, for example nuclease domain from a Type IIs endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind a 18 nucleotide recognition sequence.

(3) Guide RNA/Cas Endonuclease System

Bacteria and archaea have evolved adaptive immune defenses termed clustered regularly interspaced short palindromic repeats (CRISPR)/CRISPR-associated (Cas) systems that use short RNA to direct degradation of foreign nucleic acids (Prashant Mali et al., RNA-Guided Human Genome Engineering via Cas9 Science 339,823 (2013),). The type II CRISPR/Cas system from bacteria employs a crRNA and tracrRNA to guide the Cas endonuclease to its DNA target. The crRNA (CRISPR RNA) contains the region complementary to the DNA target and base pairs with the tracrRNA (trans-activating CRISPR RNA) forming a RNA duplex that directs the Cas endonuclease to cleave the DNA target.

As used herein, the term “guide RNA” refers to a synthetic fusion of two RNA molecules, a crRNA (CRISPR RNA) comprising a variable targeting domain, and a tracrRNA

The term “variable targeting domain” refers to a 12 to 30 nucleotide sequence 5-prime of the GUUUU sequence motif in the guide RNA that is complementary to a DNA target site in the genome of a plant cell, plant or seed.

In one embodiment of the invention the variable target domain is 12, 13, 14, 15, 15, 16, 17, 18, 19, 20, 21, 22, 23, 24, 25, 26, 27, 28, 29 or 30 nucleotides in length.

In one embodiment of the disclosure, the guide RNA comprises a cRNA and a tracrRNA of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site.

In one embodiment the guide RNA can be introduce into the plant cell directly using particle bombardment.

In another embodiment the guide RNA can be introduced indirectly by introducing a recombinant DNA molecule comprising the corresponding guide DNA sequence operably linked to a plant specific promoter that is capable of transcribing the guide RNA in said plant cell. The term “corresponding guide DNA” refers to a DNA molecule that is identical to the RNA molecule but has a “T” substituted for each “U” of the RNA molecule.

In some embodiments, the guide RNA is introduced via particle bombardment or Agrobacterium transformation of a recombinant DNA construct comprising the corresponding guide DNA operably linked to a plant U6 polymerase III promoter.

In one embodiment, the RNA that guides the RNA/Cas9 endonuclease complex: is a duplexed RNA comprising a duplex crRNA-tracrRNA. One advantage of using a guide RNA versus a duplexed crRNA-tracrRNA is that only one expression cassette needs to be made to express the fused guide RNA.

### III. Target Sites for Cas Endonucleases

The terms “target site”, “target sequence”, “target DNA”, “target locus”, “genomic target site”, “genomic target sequence” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

In one embodiments, the target site can be similar to a DNA recognition site or target site that that is specifically recognized and/or bound by a double-strand break inducing agent such as a LIG3-4 endonuclease (US Patent Application Publication Number 2009/0133152 A1, published May 21, 2009) or a MS26++meganuclease (U.S. patent application Ser. No. 13/526,912, filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide or (iv) any combination of (i)-(iii).

Methods for modifying a plant genomic target site are disclosed herein. In one embodiment, a method for modifying a target site in the genome of a plant cell comprises introducing a guide RNA into a plant cell having a Cas endonuclease, wherein said guide RNA and Cas endonuclease are capable of forming a complex that enables the Cas endonuclease to introduce a double strand break at said target site, wherein said guide RNA comprises a variable targeting domain that is complementary to said target site.

Also provided is a method for modifying a target site in the genome of a plant cell, the method comprising introducing a guide RNA and a Cas endonuclease into said plant, wherein said guide RNA and Cas endonuclease are capable of forming a complex that enables the Cas endonuclease to introduce a double strand break at said target site, wherein said guide RNA comprises a variable targeting domain that is complementary to said target site.

Further provided is a method for modifying a target site in the genome of a plant cell, the method comprising introducing a guide RNA and a donor DNA into a plant cell having a Cas endonuclease, wherein said guide RNA and Cas endonuclease are capable of forming a complex that enables the Cas endonuclease to introduce a double strand break at said target site, wherein said guide RNA comprises a variable targeting domain that is complementary to said target site, wherein said donor DNA comprises a polynucleotide of interest.

Further provided is a method for modifying a target site in the genome of a plant cell, the method comprising: a) introducing into a plant cell a guide RNA comprising a variable targeting domain that is complementary to said target site and a Cas endonuclease, wherein said guide RNA and Cas endonuclease are capable of forming a complex that enables the Cas endonuclease to introduce a double strand break at said target site and b) identifying at least one plant cell that has a modification at said target, wherein the modification includes at least one deletion or substitution of one or more nucleotides in said target site.

Further provided, a method for modifying a target DNA sequence in the genome of a plant cell, the method comprising: a) introducing into a plant cell a first recombinant DNA construct capable of expressing a guide RNA and a second recombinant DNA construct capable of expressing a Cas endonuclease, wherein said guide RNA and Cas endonuclease are capable of forming a complex that enables the Cas endonuclease to introduce a double strand break at said target site and b) identifying at least one plant cell that has a modification at said target, wherein the modification includes at least one deletion or substitution of one or more nucleotides in said target site.

The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs.

Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the double-strand break of a target site by an endonuclease are known in the art and generally measure the overall activity and specificity of the agent on DNA substrates containing recognition sites.

B. Transactivator elements

Transactivator elements are provided herein for use in regulating the expression of genes of interest by selectively activating inducible promoters. For example, the polynucleotides encoding transactivator proteins of the invention can be placed under the control of a constitutive, tissue-specific, or other transactivator-inducible promoter to control the expression of a nucleotide of interest operably linked to a transactivator-inducible promoter. In some embodiments, a polynucleotide encoding a transactivator protein can be provided on an expression cassette in a separate plant from the expression or suppression cassette comprising the corresponding transactivator-inducible promoter. Expression cassettes provided herein comprising polynucleotides encoding transactivator proteins can further comprise operably linked promoters that drive expression of the transactivator in a plant. As used herein, “transactivator A” and “transactivator B” refer to any transactivator element used for regulating the expression of genes of interest by selectively activating inducible promoters. Examples of transactivators include the GAL4DBD-VP16/UAS PRO system, the T7 polymerase/T7 PRO system and the LexA transactivator system commonly known in the art, or any combination thereof, (Yagi, et al., (2010) Proc. Natl. Acad. Sci. 107(37):16166-16171).

As used herein, “transactivator promoter” refers to a promoter operably linked to a polynucleotide encoding a transactivator. In specific embodiments, expression cassettes are provided encoding a polynucleotide encoding a transactivator operably linked to a constitutive or tissue-specific promoter. For example, the tissue-specific promoter operably linked to a polynucleotide encoding a transactivator can be an ovule-specific promoter wherein the transactivator is specifically expressed in the ovule of a plant. Such a transactivator specifically expressed in the ovule of a plant can activate the corresponding transactivator-inducible promoter resulting in the expression of a gene of interest only in the ovule. In one embodiment of the invention, a first plant comprising an expression cassette comprising a polynucleotide encoding transactivator A operably linked to an ovule-specific promoter is crossed with a second plant comprising a suppression cassette comprising a CENH3 silencing element operably linked to a transactivator A-inducible promoter. In the resulting progeny plant, the CENH3 silencing element is specifically expressed in the ovule.

In another embodiment of the invention, a first plant comprising an expression cassette comprising a polynucleotide encoding transactivator B under the control of a constitutive promoter is crossed with a second plant comprising a suppression cassette comprising a MiMe silencing element under the control of a transactivator-inducible promoter. In progeny from the resulting cross, the transactivator activates constitutive expression of the MiMe silencing element. In certain embodiments, an expression cassette comprising a polynucleotide encoding transactivator A is provided in the same plant as a suppression cassette comprising a transactivator B-inducible promoter, wherein transactivator A does not activate the expression of the transactivator B-inducible promoter.

C. Expression Cassettes and Suppression Cassettes

Compositions of the invention also encompass expression cassettes and suppression cassettes. It is recognized that the polynucleotides and silencing elements of the invention can be provided in expression cassettes and suppression cassettes, respectively, for expression in a plant of interest. Expression cassettes provided herein may comprise, for example, polynucleotides encoding a transactivator, an active CENH3 mutant, and/or wild-type CENH3, or fragments or variants thereof. Suppression cassettes provided herein may, for example, comprise a silencing element as described herein above.

The expression and suppression cassettes of the invention can include 5′ and 3′ regulatory sequences operably linked to the polynucleotide or silencing elements of the invention. “Operably linked” is intended to mean a functional linkage between two or more elements. For example, an operable linkage between a polynucleotide and a regulatory sequence (i.e., a promoter) is a functional link that allows for expression of the polynucleotide of the invention. In particular examples, a polynucleotide or silencing element of the invention can be operably linked to a promoter that drives expression in a plant. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression and suppression cassettes can be provided with a plurality of restriction sites and/or recombination sites for insertion of the polynucleotide to be under the transcriptional regulation of the regulatory regions. The expression and suppression cassettes may additionally contain selectable marker genes.

The expression and suppression cassettes can include in the 5′-3′ direction of transcription, a transcriptional and translational initiation region (i.e., a promoter), a polynucleotide encoding a polypeptide or the silencing element(s) employed in the methods and compositions of the invention, and a transcriptional and translational termination region (i.e., termination region) functional in plants. In those embodiments, where the suppression cassettes encode double stranded RNA the suppression cassette can comprise two convergent promoters that drive transcription of the operably linked silencing element. “Convergent promoters” refers to promoters that are oriented on either terminus of the operably linked silencing element such that each promoter drives transcription of the silencing element in opposite directions, yielding two transcripts. In such embodiments, the convergent promoters allow for the transcription of the sense and anti-sense strand and thus allow for the formation of a dsRNA.

The regulatory regions (i.e., promoters, transcriptional regulatory regions and translational termination regions) and/or the polynucleotides or silencing elements employed in the invention may be native/analogous to the host cell or to each other. Alternatively, the regulatory regions and/or the polynucleotides or silencing elements employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding a polypeptide or silencing element, may be native with the plant host, or may be derived from another source (i.e., foreign or heterologous) to the promoter, the polynucleotide, the silencing element, the plant host, or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression or suppression cassettes of the invention, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In particular embodiments, the silencing element of a suppression cassette may be operably linked to a promoter that drives expression of the silencing element in a plant. In other embodiments, polynucleotides encoding an active CENH3 mutant, wild-type CENH3 or transactivator of an expression cassette may be operably linked to a promoter that drives expression of the polynucleotide in a plant. It is recognized that a number of promoters can be used in the practice of the invention. Polynucleotides encoding silencing elements can be combined with constitutive, tissue-preferred, transactivator-inducible or other promoters for expression in plants.

Such constitutive promoters include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

An inducible promoter, for instance, a transactivator-inducible promoter are provided. For example, transactivator-inducible promoters for use in the expression or suppression cassettes disclosed herein include: GaI4DBD::VP16/UAS; GaI4DBD::hypothetical activator domain/UAS; T7 Polymerase/T7 promoter; other proprietary systems; in theory: unique DNA binding domain::activation domain/DNA recognition element::minimal promoter element as demonstrated in numerous novel fusions in plant transient experimental systems.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci. USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

Egg and central cell-specific promoters and central cell-specific promoters can be utilized to confine expression of silencing elements, active CENH3 mutants, or wild-type CENH3 to the central cell of a plant. For example, AT-DD45 PRO, AT-RKD1 PRO or AT-RKD2 PRO can be used as egg cell-specific promoters. The egg and central cell-specific MEA (FIS1) and FIS2 promoters are also useful reproductive tissue-specific promoters (Luo, et al., (2000) Proc. Natl. Acad. Sci. USA 97:10637-10642; Vielle-Calzada, et al., (1999) Genes Dev. 13:2971-2982). The central cell specific promoter. Other examples of egg cell and central cell-specific promoters can be found, for example, in Steffen, et al., (2007) Plant J 51: 281-292 and Ohnishi, et al., (2011) Plant Physiology 155:881-891, herein incorporated by reference in their entirety. For example, central cell specific promoters from Steffen, et al., can be used, including, for example, AT-DD7 PRO, AT-DD9 PRO, AT-DD22 PRO, AT-DD25 PRO, AT-DD36 PRO, AT-DD41 PRO, AT-DD66 PRO and AT-DD65 PRO.

Ovule-specific promoters are known and can be selected for ovule-specific expression of polynucleotides disclosed elsewhere herein. For example, ovule-specific promoters can drive expression of transactivators or active CENH3 mutants in the entire ovule, including, but not limited to the egg cell and central cell. The ovule-specific promoter for BEL1 gene can also be used (Reiser, et al., (1995) Cell 83:735-742; GenBank Accession Number U39944; Ray, et al, (1994) Proc. Natl. Acad. Sci. USA 91:5761-5765) as well as those disclosed in U.S. patent application Ser. No. 12/912,231, filed Oct. 26, 2010, herein incorporated by reference in its entirety.

Possible promoters also include the Black Cherry promoter for Prunasin Hydrolase (PH DL1.4 PRO) (U.S. Pat. No. 6,797,859), Thioredoxin H promoter from cucumber and rice (Fukuda, et al., (2005). Plant Cell Physiol. 46(11):1779-86), Rice (RSs1) (Shi, et al., (1994). J. Exp. Bot. 45(274):623-631) and maize sucrose synthese -1 promoters (Yang, et al., (1990) PNAS 87:4144-4148), PP2 promoter from pumpkin Guo, et al., (2004) Transgenic Research 13:559-566), At SUC2 promoter (Truernit, et al., (1995) Planta 196(3):564-70, At SAM-1 (S-adenosylmethionine synthetase) (Mijnsbrugge, et al., (1996) Plant. Cell. Physiol. 37(8):1108-1115) and the Rice tungro bacilliform virus (RTBV) promoter (Bhattacharyya-Pakrasi, et al., (1993) Plant J. 4(1):71-79).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol Bioeng 85:610-9 and Fetter, et al., (2004) Plant Cell 16:215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Sci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the present invention.

D. Fragments and Variants

The expression and suppression cassettes of the invention can be designed based on the naturally occurring CENH3, Spo11-1, Rec8 or OSd1 polynucleotides or fragments or variants thereof. By “fragment” is intended a portion of the nucleotide sequence. Fragments of the disclosed nucleotide sequences may range from at least about 10, 16, 20, 50, 75, 100, 150, 200, 250, 300, 350, 400, 450 or 500 contiguous nucleotides, or up to the number of nucleotides present in a full-length CENH3, Spo11-1, Rec8 or OSd1 polynucleotide disclosed herein (for example, 1089 nucleotides for SEQ ID NO: 2) so long as the fragment achieves the desired objective, i.e., expression of a biologically active polypeptide of interest (for example, the active CENH3 mutant or CENH3 polypeptide) or expression of a functional silencing element that suppresses expression or function of the CENH3, Spo11-1, Rec8 or OSd1 polypeptide.

By “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide comprises a naturally occurring nucleotide sequence, for example, a naturally occurring CENH3, Spo11-1, Rec8 or OSd1 polynucleotide. For polynucleotides, naturally occurring variants can be identified with the use of well-known molecular biology techniques such as, for example, polymerase chain reaction (PCR) and hybridization techniques as outlined elsewhere herein. Variant polynucleotides also include synthetically derived polynucleotides, such as those generated, for example, by using site-directed mutagenesis. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters commonly known in the art.

In particular embodiments, a silencing element of the invention may comprise the full-length nucleotide sequence of SEQ ID NOS: 2, 3, 4 and/or 5 or a fragment of the nucleotide sequence of SEQ ID NOS: 2, 3, 4 and/or 5. Additionally, silencing elements of the invention may comprise a variant of the full-length nucleotide sequence of SEQ ID NOS: 2, 3, 4 and/or 5 or a variant of a fragment of the nucleotide sequence of SEQ ID NOS: 2, 3, 4 and/or 5. Such variants will maintain at least 80% sequence identity to the nucleotide sequence of the native full-length sequence or fragment from which the variant is derived. It is recognized that the CENH3 and active CENH3 mutants can be altered in various ways including amino acid substitutions, deletions, truncations, and insertions. Methods for such manipulations are generally known in the art. Nucleotide sequence variants and fragments of the CENH3,

Rec8 or OSd1 gene can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

Thus, the expression and suppression cassettes can be based on the naturally occurring nucleotide sequences as well as variations and modified forms thereof. Such variants will continue to possess the desired activity. Obviously, where a functional polypeptide is to be expressed, the mutations that will be made in the DNA encoding the variant polypeptide must not place the sequence out of reading frame and optimally will not create complementary regions that could produce secondary mRNA structure. See, EP Patent Application Publication Number 75,444.

The deletions, insertions and substitutions of the encoded polypeptides encompassed herein are not expected to produce radical changes in the characteristics of the protein. However, when it is difficult to predict the exact effect of the substitution, deletion or insertion in advance of doing so, one skilled in the art will appreciate that the effect will be evaluated by routine screening assays. Deletions, insertions and substitutions within a polynucleotide of interest are made such that the variant polynucleotide retains the desired activity, i.e., encoding a functional CENH3 variant, or encoding a functional silencing element that effectively suppresses expression or function of the CENH3, Spo11-1, Rec8 or OSd1 polypeptide. In an inbred situation, analyses of protein functionality would be best done through cytogenetic evaluations, i.e. microscopy of meiotic stages and resultant products. Mis-function of these proteins would have impacts on fertility and offspring health (across reasonable numbers of plants) which would be in most cases readily noticed. In crosses between differing genetic backgrounds, molecular markers could be used to assess recombination and segregation.

### III. Plants

Plants, plant cells, plant parts and seeds and grain comprising one or more of the expression cassettes and suppression cassettes described elsewhere herein are provided. In specific embodiments, the plants and/or plant parts comprise stably incorporated in the genome at least one transactivator expression cassette, at least one active CENH3 mutant expression cassette, at least one wild-type CENH3 expression cassette, at least one MiMe suppression cassette, and/or at least one wild-type CENH3 suppression cassette. Thus, the invention provides plants, plant cells, plant parts and seed that have stably incorporated into their genome a transactivator A expression cassette, an active CENH3 mutant expression cassette and a MiMe suppression cassette. Further provided are plants, plant cells, plant parts and seeds that have stably incorporated into their genome a transactivator B expression cassette, a wild-type CENH3 expression cassette and a wild-type CENH3 suppression cassette. In specific embodiments, progeny plants are provided resulting from the cross of a plant having stably incorporated into the genome a transactivator A expression cassette, an active CENH3 mutant expression cassette and a MiMe suppression cassette with a plant having stably incorporated into the genome a transactivator B expression cassette, a wild-type CENH3 expression cassette and a wild-type CENH3 suppression cassette wherein the progeny plant is a self-reproducing hybrid plant. Such self-reproducing hybrid progeny plants comprise at least one transactivator expression cassette, at least one active CENH3 mutant expression cassette, at least one wild-type CENH3 expression cassette, at least one MiMe suppression cassette and/or at least one wild-type CENH3 suppression cassette.

In specific embodiments, plants and seeds are provided comprising a suppression cassette comprising a MiMe silencing element operably linked to a transactivator B-inducible promoter, an expression cassette comprising a polynucleotide encoding an active CENH3 mutant operably linked to an ovule-specific promoter, and an expression cassette comprising a polynucleotide encoding a transactivator A operably linked to an ovule-specific promoter. In other embodiments, plants and seeds are provided comprising a suppression cassette comprising a wild-type CENH3 silencing element operably linked to a transactivator A-inducible promoter, an expression cassette comprising a polynucleotide encoding a wild-type CENH3 polypeptide operably linked to an egg-cell specific promoter, and an expression cassette comprising a polynucleotide encoding a transactivator B operably linked to a promoter.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the invention, provided that these parts comprise the introduced polynucleotides.

The expression cassettes and suppression cassettes disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus ellioth), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis) and Poplar and Eucalyptus. In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments soybean plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In some embodiments, the polynucleotides comprising the expression cassettes or suppression cassettes described elsewhere herein are engineered into a molecular stack. Thus, the various plants, plant cells and seeds disclosed herein can further comprise one or more traits of interest, and in more specific embodiments, the plant, plant part or plant cell is stacked with any combination of polynucleotide sequences of interest, expression cassettes of interest, or suppression cassettes of interest in order to create plants with a desired combination of traits. As used herein, the term “stacked” includes having the multiple traits present in the same plant.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

Thus, in specific embodiments, the expression cassettes and suppression cassettes disclosed herein function to produce self-reproducing hybrid progeny plants when combined in a progeny plant. Such expression and suppression cassettes can then be stacked with any other sequence of interest, including polynucleotides conferring herbicide tolerance. Non-limiting examples of such sequences are disclosed elsewhere herein.

A “subject plant or plant cell” is one in which genetic alteration, such as transformation, has been affected as to a polynucleotide of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The methods of the invention comprise introducing expression and suppression cassettes disclosed herein into the genome of a plant or plant cell. The methods provided herein do not depend on a particular method for introducing polynucleotides comprising the expression or suppression cassettes into the host cell, only that the polynucleotide gains access to the interior of at least one cell of the host. Methods for introducing polynucleotides into host cells (i.e., plants) are known in the art and include, but are not limited to, stable transformation methods, transient transformation methods, and virus-mediated methods.

“Stable transformation” is intended to mean that the nucleotide construct introduced into a host (i.e., a plant) integrates into the genome of the plant and is capable of being inherited by the progeny thereof. “Transient transformation” is intended to mean that a polynucleotide is introduced into the host (i.e., a plant) and expressed temporally.

Transformation protocols as well as protocols for introducing polynucleotide sequences into plants may vary depending on the type of plant or plant cell, i.e., monocot or dicot, targeted for transformation. Suitable methods of introducing polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (Townsend, et al., U.S. Pat. No. 5,563,055; Zhao, et al., U.S. Pat. No. 5,981,840), direct gene transfer (Paszkowski, et al., (1984) EMBO J. 3:2717-2722) and ballistic particle acceleration (see, for example, Sanford, et al., U.S. Pat. No. 4,945,050; Tomes et al., U.S. Pat. No. 5,879,918; Tomes, et al., U.S. Pat. No. 5,886,244; Bidney, et al., U.S. Pat. No. 5,932,782; Tomes, et al., (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 2000/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); Tomes, U.S. Pat. No. 5,240,855; Buising, et al., U.S. Pat. Nos. 5,322,783 and 5,324,646; Tomes, et al., (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; Bowen, et al., U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens); all of which are herein incorporated by reference.

In specific embodiments, the expression and suppression cassettes disclosed herein can be provided to a plant using a variety of transient transformation methods. Such transient transformation methods include, but are not limited to, the introduction of the expression and suppression cassettes directly into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, expression and suppression cassettes can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In other embodiments, expression and suppression cassettes disclosed herein may be introduced into plants by contacting plants with a virus or viral nucleic acids. Generally, such methods involve incorporating a nucleotide construct of the invention within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the invention can be contained in transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having expression and suppression cassettes disclosed herein, stably incorporated into their genome.

### IV. SRH Cassette Insertion Location

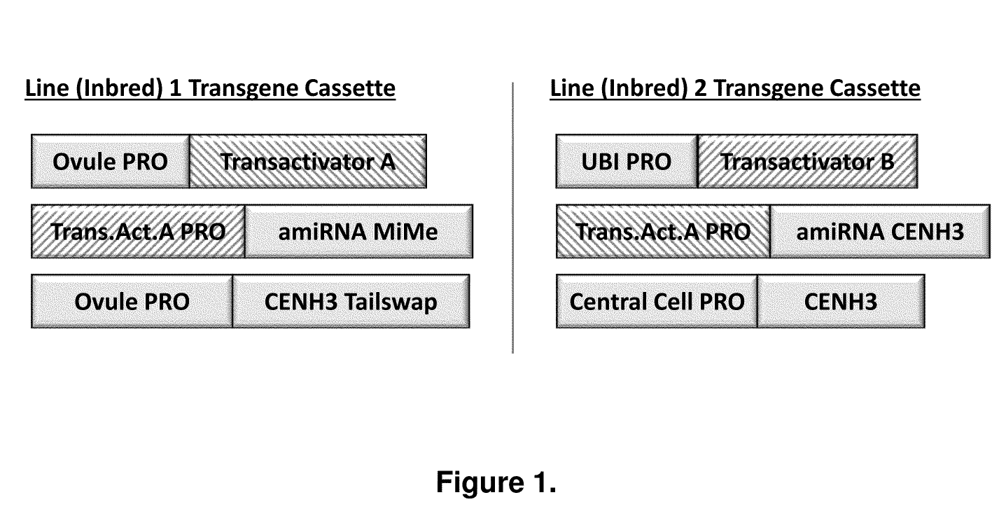

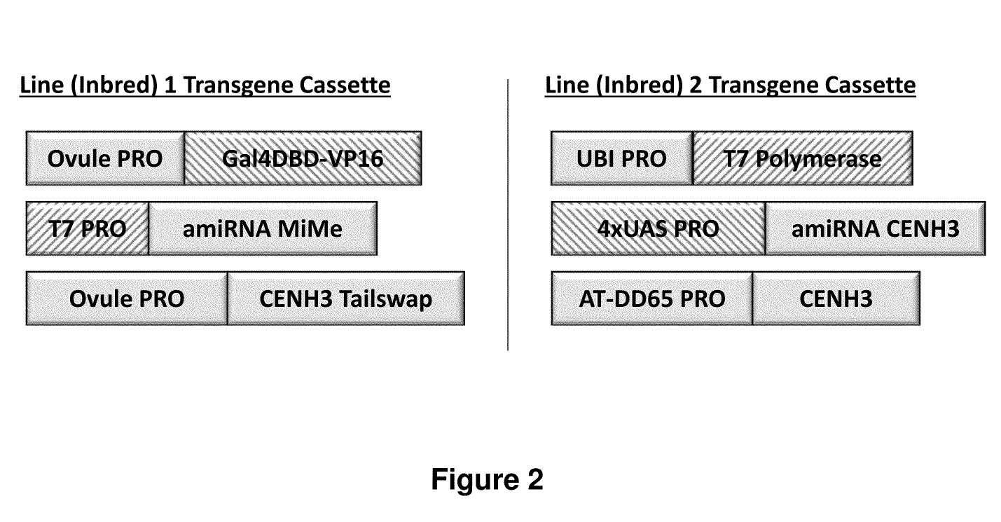

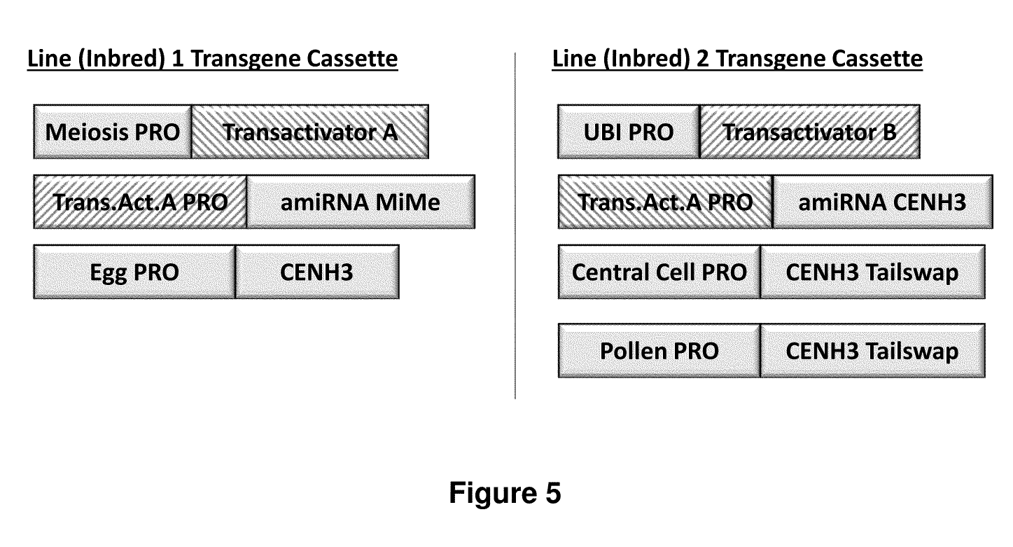

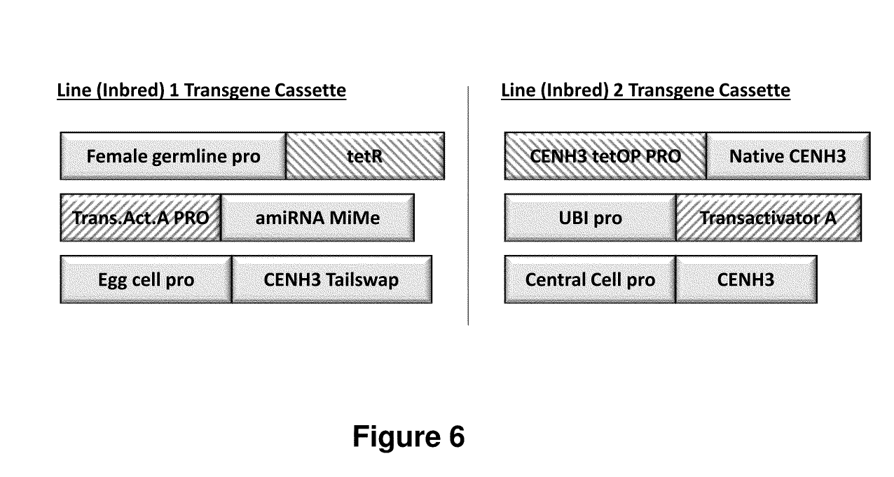

Methods are known to insert polynucleotides at specific locations in the plant genome, including but not limited to SSI, Cas9, TALENs, meganucleases or other DSB technologies. These methods may be used to insert a self-reproducing hybrids cassette, for example, those in FIGS. 1, 2, 5, 6, & 7, into or next to a MiMe, Genome Elimination, or parthenogenesis locus. As used herein a MiMe, Genome Elimination, or parthenogenesis locus refers to a dominant or recessive allele of a gene responsible for one of these traits or a portion thereof. The inserted cassette may partially or completely complement the allele in some or all contexts.

For example, in one method, a CENH3 knockout may be created or targeted for nearby insertion using one of these technologies. Such a CENH3 knockout could be recessive. The allele may be maintained as a heterozygote or as a homozygote if complemented by a transgene cassette. In some instances the transgene cassette would be a complete or partial SRH cassette. In some instances the SRH cassette would be inserted at or near the CENH3 locus. If the SRH cassette is located at or near the CENH3 locus, then the combined locus/cassette would segregate and function as a single locus. In the situation of a recessive allele, both parents in the hybrid cross would need to contain recessive alleles at the native locus. This would alleviate a multi-locus trait that would otherwise hinder self-reproducing hybrid production using recessive or native trait loci. In some examples, both parents may contain complementary cross-activating SRH cassettes at or near the recessive native locus. In this way, trait introgression would be simplified and limit transgenic drag in many genetic backgrounds.

### EXAMPLES

### Example 1

### Plant Material and Growth Conditions

Plants were grown in artificial soil mix at 20° C. under fluorescent lighting. Wild-type and mutant strains of Arabidopsis were obtained from ABRC, Ohio or NASC, UK. dyad was crossed to the No-0 strain to generate populations that were heterozygous for markers across the genome. MiMe plants were a mixture of Col-0 from Atspo11-1-3/Atrec8-3 and No-0 from osd1-1 (S1). The GEM plants used in this study are F1 progeny obtained by crossing cenh3-1/cenh3-1 GFP-tailswap/GFP-tailswap (female) to cenh3-1/cenh3-1 GFPCENH3/GFP-CENH3 (male).

cenh3-1 was isolated by the TILLING procedure (Comai & Henikoff, (2006) Plant J 45:684-94). The TILLING population was created by mutagenizing Arabidopsis thaliana in the Col-0 accession with ethylmethanesulfonate, using standard protocols. Cenh3-1 was isolated by TILLING using the CEL1 heteroduplex cleavage assay, with PCR primers specific for the CENH3/HTR12 gene.

To cross wild-type as the female to GFP-tailswap as the male, a dissecting microscope was used to directly observe pollen deposition on the stigma (GFP-tailswap is mostly male-sterile). The amount of viable pollen in individual flowers of GFP-tailswap varies. Flowers that clearly showed higher amounts of pollen were selected and pollinated with more than 60 anthers (10 GFP-tailswap flowers) per wild-type stigma to achieve the seed set reported in Table 1. Using an optivisor (magnifying lens) and approximately 12 anthers (2 GFP-tailswap flowers) per wild-type stigma, a much lower seed set per silique was obtained. Seed from GFP-tailswap×wild-type crosses were sown on 1×MS plates containing 1% sucrose to maximize germination efficiency, particularly of seed that had an abnormal appearance. Late germinating seeds were frequently haploid.

A chimera was created in which the A. thaliana CENH3 tail from CENH3 is replaced with the CENH3 tail domain from maize (Zea mays), thereby generating a fusion of the maize CENH3 tail and A. thaliana CENH3 histone-fold domain, and transformed the fusion into cenh3-1 heterozygotes. As expected, this GFP-maize tailswap protein was targeted to kinetochores and rescued the embryo-lethal phenotype of cenh3-1.

### Example 2

### Genotypina and Microsatellite Marker Analysis

Primers for osd1-1, Atspo11-1-3 and Atrec8-3 (MiMe) genotyping are described (S1).

Microsatellite markers were analyzed. Primer sequences were obtained from TAIR (www.Arabidopsis.org) or from the MSAT database (INRA). cenh3-1: a point mutation G161A in the CENH3 gene (also known as HTR12) detected with dCAPS primers (dCAPs restriction polymorphism with EcoRV, the wild-type allele cuts):

Primer 1:
(SEQ ID NO: 6)
GGTGCGATTTCTCCAGCAGTAAAAATC
Primer 2:
(SEQ ID NO: 7)
CTGAGAAGATGAAGCACCGGCGATAT

Detection of GFP-tailswap insertion on chromosome 1:

Primer 1 for wild-type and T-DNA:
(SEQ ID NO: 8)
CACATACTCGCTACTGGTCAGAGAATC
Primer 2 for wild-type only:
(SEQ ID NO: 9)
CTGAAGCTGAACCTTCGTCTCG
Primer 3 for the T-DNA:
(SEQ ID NO: 10)
AATCCAGATCCCCCGAATTA

Primers for detection of GFP-CENH3:

(SEQ ID NO: 11)
CAGCAGAACACCCCCATC (in GFP)
(SEQ ID NO: 12)
CTGAGAAGATGAAGCACCGGCGATAT (in CENH3)

### Ploidy Analysis

MiMe and osd1 offspring ploidy analyses were performed by flow cytometry and systemically confirmed by chromosome spreads. For dyad offspring, ploidy analysis was by flow cytometry and randomly selected diploid eliminants (n=5) were further confirmed by FISH analysis using a centromere repeat probe to count chromosomes and all were found to be diploids. Isolation of nuclei for flow cytometry was performed. Flow cytometry analysis was carried out using an internal diploid and tetraploid control to unambiguously identify diploid plants.

In elimination crosses to the wild-type tetraploid line (C24 background), triploids were identified as late flowering (due to combination of the Col-0 FRIGIDA and C24 FLOWERING LOCUS C alleles). The aneuploid plants show distinct morphological phenotypes such as altered vegetative growth, variation in rosette leaf morphology (size and shape), a range of leaf color (pale yellow to dark green) and thus can be easily distinguished from normal diploid wild-type plants. Further, aneuploid plants show varied flowering time and mostly have reduced fertility and seed set. Putative diploids were genotyped for at least one marker per chromosome (Chr 1: F511, CIW12; Chr 2: MSAT2.11; Chr 3: MSAT3.19, CIW11; Chr 4: nga8; Chr 5: CTR1.2, nga106). Eliminants were identified as having only C24 alleles, in addition to lacking GFP fluorescence at the centromeres which is present in the GEM line. Random diploid plants (n=8) were further confirmed by karyotyping in meiotic chromosome spreads and all were found to be diploids.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for producing a self-reproducing hybrid plant comprising:
a) obtaining a first plant comprising in its genome a first suppression cassette and a first expression cassette,
i) wherein said first suppression cassette comprises at least one first silencing element wherein said first silencing element, when expressed by said self-reproducing hybrid plant, reduces the level of at least one target sequence, wherein said target sequence comprises a member selected from the group consisting of,
A) a gene critical to meiotic second division reduction,
B) a gene critical to meiotic recombination, and
C) a gene critical to meiotic chromosome segregation,
ii) wherein the first plant comprises in the first suppression cassette or in a second suppression cassette an additional silencing element that inhibits wild-type centromere-specific polypeptide activity; and
iii) wherein said first expression cassette comprises a nucleic acid molecule encoding an active centromere-specific mutant polypeptide that is only active in said self-reproducing hybrid plant;
b) obtaining a second plant comprising in its genome a repressor cassette and a second expression cassette,
i) wherein said repressor cassette comprises a modified native repressible promoter linked to a wild-type centromere-specific gene, repressed in said self-reproducing hybrid plant, reducing the level of a wild-type centromere-specific polypeptide or a homolog thereof;
ii) wherein said second expression cassette comprises a transactivator and a nucleic acid molecule encoding a wild-type centromere-specific polypeptide or homolog thereof, wherein said centromere-specific polypeptide is expressed in said self-reproducing hybrid plant; and
c) crossing said first plant with said second plant thereby producing said self-reproducing hybrid plant.

2. The method of claim 1, wherein the active centromere-specific mutant polypeptide is CENH3, CENPC, MCM21, MIS12, NDC80 and NUF2.

3. The method of claim 1, comprising at least one first silencing element, wherein said at least one first silencing element has inhibitory activity against a target sequence, wherein said target sequence comprises a member selected from the group consisting of:
a) Osd1 or a homolog thereof;
b) Spo11-1 or a homolog thereof; and
c) Rec8 or a homolog thereof.

4. The method of claim 1, wherein an inducible promoter is operably linked to the at least one silencing element.

5. The method of claim 1, wherein the additional silencing element targets the promoter driving the wild-type centromere-specific polypeptide, or a homolog thereof.

6. The method of claim 1, wherein the additional silencing element targets (a) the nucleic acid encoding the wild-type centromere-specific polypeptide or homolog thereof or (b) wild-type centromere-specific polypeptide or homolog thereof.

7. The method of claim 1, wherein the additional silencing element is a repressor system.

8. (canceled)

9. The method of claim 1, wherein the wild-type centromere-specific polypeptide is CENH3 or a homolog thereof.

10. The method of claim 1, wherein the nucleic acid molecule encoding an active centromere-specific mutant polypeptide is CENH3-tailswap.

11. The method of claim 1, wherein a promoter is operably linked to the additional silencing element, and the promoter is specifically induced by a transactivator.

12. (canceled)

13. (canceled)

14. A first plant comprising an active CENH3 mutant expression cassette comprising a central cell-specific promoter, a CENH3 suppression cassette comprising a transactivator A-inducible promoter, a CENH3 expression cassette comprising an egg-cell specific promoter, and a transactivator B expression cassette comprising an active promoter.

15. A second plant comprising an active CENH3 mutant expression cassette comprising a pollen or sperm-cell expressing promoter, a MiMe suppression cassette comprising a transactivator B-inducible promoter, and a transactivator A expression cassette comprising a germline preferred promoter.

16. A pair of plants to produce a tetraploid zygote wherein the first plant comprises an active CENH3 mutant expression cassette comprising a central cell-specific promoter, a CENH3 suppression cassette comprising a transactivator A-inducible promoter, a CENH3 expression cassette comprising an egg-cell specific promoter, and a transactivator B expression cassette comprising an active promoter and wherein the second plant comprises an active CENH3 mutant expression cassette comprising a sperm-cell preferred promoter, a MiMe suppression cassette comprising a transactivator B-inducible promoter, and a transactivator A expression cassette comprising a germline preferred promoter.

17. A method for producing a tetraploid zygote comprising:
(a) crossing a first plant comprising an active CENH3 mutant expression cassette comprising a central cell-specific promoter, a CENH3 suppression cassette comprising a transactivator A-inducible promoter, a CENH3 expression cassette comprising an active promoter, and a transactivator B expression cassette comprising an ovule-specific promoter with a second plant comprising an active CENH3 mutant expression cassette comprising a sperm-cell preferred promoter, a MiMe suppression cassette comprising a transactivator B-inducible promoter, and a transactivator A expression cassette comprising a germline preferred promoter to produce a tetraploid zygote

[symbol]

18. The method of claim 17, where in the tetraploid zygote subsequently loses the male genome from the sperm cell following a generation of self-fertilization, ultimately resulting in a self-reproducing hybrid progeny plant.

19. (canceled)

20. (canceled)

21. (canceled)

22. (canceled)

23. A method for providing self reproducing hybrids comprising:
a. Providing a first inbred plant line which comprises:
i. transactivator elements A and B
b. Crossing the first inbred plant line with a second inbred line which comprises
i. Promoter A linked to MiMe silencing element; and
ii. Promoter B linked to CENH3 silencing element.

24. The method of claim 23 for providing self reproducing hybrids comprising:
a. Providing a first inbred plant line which comprises:
i. A female germline promoter linked to a repressor,
ii. a transactivator A promoter linked to MiMe, and
iii. an egg cell promoter linked to CENH3 tailswap
b. Crossing the first inbred plant line with a second inbred line which comprises:
i. a CENH3 tetOP promoter linked to native CENH3,
ii. a constitutive promoter linked to transactivator A, and
iii. a central cell promoter linked to CENH3
c. Producing an F1 hybrid from the two parent lines, where the 2 component transcriptional activator and repressor are brought into a common hybrid genome and activate the silencing elements and or repress the genes required for MiMe and genome elimination.

25. The method of claim 23 for providing self reproducing hybrids comprising:
a. Providing a first inbred plant line which comprises:
i. a meiosis promoter linked to a (tetR) repressor,
ii. a transactivator A promoter linked to MiMe, and
iii. an egg cell promoter linked to CENH3.
b. Crossing the first inbred plant line with a second inbred line which comprises:
i. a CENH3 tetOP promoter linked to native CENH3,
ii. a constitutive promoter linked to transactivator A,
iii. a central cell promoter linked to CENH3 tailswap, and
iv. a pollen promoter linked to CENH3 tailswap.
c. Producing an F1 hybrid from the two parent lines, where the 2 component transcriptional activator and repressor are brought into a common hybrid genome and activate the silencing elements and or repress the genes required for MiMe and genome elimination.

26-36. (canceled)
